(19) United States
(12) Patent Application Publication
(10) Pub. No.: US 2004/0241436 A1
(43) Pub. Date: Dec. 2, 2004

(54) Nano-porous fibers and protein membranes

(75) Inventors: You-Lo Hsieh, Davis, CA (US); Jiangbing Xie, Irvine, CA (US); Yuhong Wang, Davis, CA (US); Hong Chen, Ann Arbor, MI (US); Lei Li, Davis, CA (US); Lifeng Zhang, Davis, CA (US); Corine Cecile, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/761,660

(22) Filed: Jan. 20, 2004

Related U.S. Application Data
(63) Continuation of application No. PCT/US03/36307, filed on Nov. 12, 2003.
(60) Provisional application No. 60/447,879, filed on Feb. 14, 2003.
(60) Provisional application No. 60/425,948, filed on Nov. 12, 2002.

(51) Int. Cl. A61F 2/02; B32B 27/04; B32B 27/12

(52) U.S. Cl. 428/361; 623/23.76; 435/398; 623/23.74; 435/399; 442/123; 428/375; 428/376

(57) ABSTRACT

The present invention provides nano-porous fibers and protein membrane compositions. In certain embodiments, continuous fiber compositions are provided having nanometer sized diameters and surface pores. In another embodiment, a protein membrane composition is provided comprising a protein; and a polymer, wherein the protein and the polymer are electrospun to form a protein membrane composition. In certain instance, the protein is covalently bound to the fiber.

### CROSS-REFERENCES TO RELATED APPLICATIONS

The present application is continuation of PCT Application No. PCT/US03/36307, filed Nov. 12, 2003 which claims priority to U.S. Provisional Patent Application No. 60/425,948, filed on Nov. 12, 2002 and U.S. Provisional Patent Application No. 60/447,879, filed on Feb. 14, 2003, the disclosures of each of which are incorporated herein by reference in their entirety for all purposes.

### BACKGROUND OF THE INVENTION

Nanofibers have been identified as potentially useful for diverse applications in which a high surface area is important. Various methods for generating nanofibers are described in the art. For example, electrospinning has been used to form fine fibers from a wide range of polymers including, e.g., polyethylene oxide (PEO), polyaramids, polyaniline, and polyethylene terephthalate (PET), (see, U.S. Pat. Nos. 4,043,331 and 5,522,879; WO 02/100,628; Doshi et al., J Electrostatics; 35:151-160 (1995); Reneker et al., I. Nanotechnology, 7:216 223 (1996); Deitzel et al., NCB. Polymer, 42:261 272 (2001); and Buchko et al., Polymer, 40:7397 7407 (1999)).

Textiles comprising nonwoven electrospun fibers have also been generated. Deitzel et al., supra, describe nonwoven textiles comprising polyethylene oxide (PEO)-based nanofibers, and Kim et al. describe melt electrospun PET and polyethylene naphthalate (PEN) (see, Kim et al., Polym. J., 32:616 618 (2000)). Electrospun fibers have also been incorporated into elastomeric membranes for protective clothing, i.e., for clothing resistant to chemical and biological warfare agents (see, Gibson et al., Coll. Surf., 188:469-481 (2001)). Electrospinning has also been used to fabricate ultrathin conductive fibers from polyaniline/PEO blends (see, Norris et al., Synt. Meta., 114: 109-114 (2000)). Others have produced micrometer fibers by electrospinning poly(L-lactide) (PLLA) and polycarbonate (PC) from volatile solvents such as dichloromethane (see, Bognitzki et al., Adv. Mater., 13:70-72 (2001)). The rapid evaporation of the volatile solvent during electrospinning yielded regular pores and pits about 100 nm on fibers. Electrospun fibers have also been used as reinforcing materials in composites. The reinforcing effects of electrospun fibers to epoxy resin (see, Bergshoef et al., Adv. Mater., 1362-1365 (1999); Kim et al., Polym. Comp., 20:124 131 (1999)) and SBR rubber (Kim et al., supra) were demonstrated. Transparent composites were prepared by applying the electrospun nylon 4, 6 fiber to an epoxy matrix (Bergshoef et al., supra). Electrospun nanofibers have also been used as templates for fabricating small scale hollow fibers in a tube by a fiber template process (“TUFT”). Caruso et al. gel coated electrospun poly(L-lactide) fibers with titanium dioxide and removed the polymer to generate hollow titanium fibers with walls of less than 200 nm and diameters ranging from hundreds of nanometers to a few micrometers (see, Adv. Mater., 13:1577-1579 (2001)).

DNA fibers (see, Fang et al., J Macromol. Sci. Phys., B36:169-173 (1997)) and metal organic polymer fibers (see, Lu et al., Inorg. Chem., 40:4516-4517 (2001)) have been produced using electrospinning. Polystyrene fibers with α-chymotrypsin attached have also been produced by electrospinning (see, Jia et al., Biotechnol. Prog. 18:1027-1032 (2002)). However, these polystyrene fibers do not have nanopores and the α-chymotrypsin is attached to functionalized fibers after electrospinning.

Many biological and chemical method are enhanced by using materials with a high specific surface area. For example, in methods where a biological material is immobilized or attached to a solid support, an increased surface area for the solid support can increase the efficiency of the methods because more biological material is available to participate in the reactions. Therefore, materials with high specific surface areas that are accessible to the reactants and products of the reactions are desirable.

In view of the foregoing, there is a need in the art for polymer-based ultra-high surface area nanofibers having even greater surface areas and compositions comprising such nanofibers. There is also a need in the art for nanofiber compositions with high surface areas comprising biological materials, wherein the biological material is immobilized on the nanofiber such that the activity of the biologcal material is maintained. The present invention satisfies these and other needs.

### SUMMARY OF THE INVENTION

The present invention provides polymer-based nanofibers having nanopores and, in a preferred embodiment, at least one biological material. The nanofibers described herein possess an ultra-high surface area due to the plurality of nanometer-size pores (i.e., nanopores) present on each nanofiber. The invention further provides polymer-based nanofibers having nanopores and biological materials, wherein the biological material is immobilized on the nanofiber such that the activity of the biological material is maintained.

One embodiment of the invention provides a nanofiber having a plurality of nanopores and comprising a first polymer (e.g., a synthetic polymer or a naturally occurring polymer) and a biological material. Suitable synthetic polymers include, for example, poly(ethylene oxide), poly(vinyl alcohol), poly(ethylene naphthalate), polyaniline, polyacrylic acid, polyacrylon nitrile, polystyrene, polymethylmethacrylate, poly(N-isopropylacrylamide), polyvinyl acetate, and derivatives or combinations thereof. Suitable naturally occurring polymer include, for example, polysaccharides, polypeptides, cellulose, poly-L-lactide, cellulose, casein, and derivatives or combinations thereof. The biological material and the first polymer can be present in a ratio of about 1:20 to about 20:1, about 1:10 to about 10:1, about 1:5 to about 5:1, about 1:4 to about 4:1, or about 1:1. In some embodiments, the biological material is incorporated into the nanofiber. In other embodiments, the biological material is covalently attached to the nanofiber via a linker (e.g., polyethylene glycol (PEG), polyacrylic acid (PAA), polyacrylamide (PAM), dimethylaminoethyl methacrylate (DMAEMA) and combinations thereof). The nanofibers typically range in diameter from about 50 nm to about 1000 nm, about 5 nm to about 500 nm, about 25 nm to about 100 nm, about 5 nm to about 25 nm, or about 10 nm to about 50 nm. The nanofibers are typically insoluble (i.e., in an aqueous solution or solvent or in an organic solution or solvent). The nanofibers may also comprise a second polymer, wherein the first and second polymers can be present in a ratio of about 1:20 to about 20: 1, about 1:10 to about 10:1, about 1:5 to about 5:1, about 1:4 to about 4:1, or about 1:1. In some embodiments, the first polymer is a synthetic organic polymer and the second polymer is a naturally occurring polymer. The polymers in the nanofibers may be crosslinked. In some embodiments, the biological material is a protein (e.g., an integral membrane protein, a structural proteins, an intracellular protein or an enzyme (e.g., a lipase, a carbohydrolase, a DNAse, or a protease). In some embodiments, the nanofibers are in a membrane or fabric.

The invention further comprises an insoluble nanofiber comprising a polymer and a biological material, wherein the nanofiber is insoluble in an aqueous solution or an organic solution.

These and other objects, features and advantages will become more apparent when read with the detailed description, examples, and figures which follow.

### DESCRIPTION OF THE DRAWINGS

FIG. 1: FIG. 1 shows electrospun membranes from PEO/casein solutions at a concentration of 5% (Table 1a) for (a)100:0; (b)80:20; (c)50:50; (d)20:80; (e)5:95; and at 10% (Table 1b) for (f)20:80.

FIG. 2: FIG. 2 shows electrospun membranes from 10% PVA/casein solutions (Table 1c) for (a)100:0; (b)70:30; (c)50:50; and (d)30:70.

FIG. 3: FIG. 3 shows crosslinked ES membranes of (a) 50:50 PEO/casein, 5%; (b) 20:80 PEO/casein, 10%; (c) 50:50 PVA/casein, 10%; and (d) a chymotrypsin (Table 3b) digested for 25 hr.

FIG. 4: FIG. 4 illustrates data showing DSC analysis of PEO/casein membranes: (a) 100:0, 5% (cast); (b) 100:0, 5%; (c) 80:20, 5%; (d) 50:50, 5%; (e) 20:80, 5%; (f) 20:80, 10%; and (g) casein neat powder.

FIG. 5: FIG. 5 illustrates data showing DSC analysis of 10% PVA/casein membranes: (a) 100:0 (cast); (b) 100:0; (c) 70:30; (d) 50:50; (e) 30:70; and (f) casein powder.

FIG. 6: FIG. 6 illustrates data showing thermogravimetric (TGA) and their derivative (dTGA) analysis of PEO/casein ES membrane (Table 1a) for (a) casein neat powder; (b) 20:80; (c) 50:50; (d) 80:20; and (e) 100:0.

FIG. 7: FIG. 7 illustrates data showing weight loss of PEO (square) and weight of residue (diamond) from TGA versus the PEO composition in solutions.

FIG. 8: FIG. 8 illustrates data showing hydrolysis of olive oil by enzyme encapsulated membranes (Diamond: PVA:lipase=80:20 (Table 1e); Square: PEO:casein:lipase=30:40:30 (Table 1d))

FIG. 9: FIG. 9 illustrates data showing the catalytic activity of enzyme carrying membranes (left 4: PVA/lipase (Table 3b, 80:20); right 2: PEO/casein/lipase (Table 3a, 30:40:30)).

FIG. 10: FIG. 10A shows reactive groups on proteins that can be used for attachment of the proteins to the nanofibers described herein. FIG. 10B shows different reactions with protein amino groups that can be used to attach proteins to the nanofibers described herein.

FIG. 11: FIG. 11 illustrates a reaction to attach an amphiphilic spacer (PEG) to cellulose.

FIG. 12: FIG. 12 illustrates data showing the total ester and carboxyl acid in cellulose and PEG-cellulose nanofiber membranes.

FIG. 13: FIG. 13 illustrates a reaction to couple a protein amino group to a PEG-cellulose nanofiber via a carbodiimide linker.

FIG. 14: FIG. 14 illustrates data showing the activity of lipase covalently bound to cellulose nanofiber membranes via a PEG spacer under various coupling reaction conditions.

FIG. 15: FIG. 15 llustrates data showing the activity of lipase covalently bound to cellulose nanofiber membranes via a PEG spacer under various binding conditions and PEG lengths.

FIG. 16: FIG. 16A shows the stability of lipase covalently bound to cellulose nanofiber membranes when exposed to various organic solvents. FIG. 16B shows the reusability of cellulose membranes with covalently bound lipase.

FIG. 17: FIG. 17 shows the properties of various cellulose nanofiber membranes generated by electrospinning of cellulose acetate.

FIG. 18: FIG. 18 shows the properties of various nanofibers: cellulose acetate vs. cellulose vs. methyacrylated cellulose.

FIG. 19: FIG. 19 illustrates a reaction to generate polyacrylic acid brushes on cellulose nanofibers by FR polymerization.

FIG. 20: FIG. 20A illustrates data showing the activity of lipase adsorbed onto polyacrylic acid brushes by FR polymerization. FIG. 20B illustrates data showing the activity of lipase adsorbed onto cellulose nanofibers by ceric ion initiation.

FIG. 21: FIG. 21 illustrates a reaction to generate polyacrylic acid brushes on cellulose nanofibers by ceric ion initiation.

FIG. 22: FIG. 22A illustrates data showing the activity of lipase adsorbed onto cellulose nanofibers by ceric ion initiation. FIG. 22B illustrates data showing the activity over time of lipase adsorbed onto cellulose nanofibers by ceric ion initiation.

FIG. 23: FIG. 23 illustrates data showing the activity of lipase incorporated into PVA nanofibers and PEO:casein nanofibers.

FIG. 24: FIG. 24 illustrates data showing the viscosity of lipase PVA solutions.

FIG. 25: FIG. 25 illustrates data showing the thermal properties of lipase:PVA membranes.

FIG. 26: FIG. 26A illustrates data showing the lipase activity of lipase incorporated into PVA nanofiber membranes. FIG. 26B illustrates data showing the lipase activity of lipase incorporated into PVA nanofiber membranes and exposed to different crosslinking pH.

FIG. 27: FIG. 27A illustrates surface grafting of polyelectrolytes onto nanofiber membranes and subsequent enzyme adsorption onto the nanofibers. FIG. 27B illustrates attachmet of PEG-diacylchloride to nanofiber membranes and subsequent covalent binding of enzyme to the nanofibers.

FIG. 28: FIG. 28 illustrates data showing the effect of carboxylic acid on the activity of lipase bound to nanofiber membranes. FIG. 28A illustrates data from lipase adsorbed onto PAA grafted cellulose nanofiber membranes. FIG. 28B illustrates data from lipase covalently bonded to PEG grafted cellulose nanofiber membranes.

FIG. 29: FIG. 29 illustrates data showing the activity of free lipase and lipase bound to cellulose nanofiber membranes when exposed to a variety of organic solvents.

FIG. 30: FIG. 30 illustrates data showing the activity of free lipase and lipase bound to cellulose nanofiber membranes at different temperatures.

FIG. 31: FIG. 31 illustrates data showing the activity of free lipase and lipase bound to cellulose nanofiber membranes at different pH.

FIG. 32: FIG. 32 illustrates data showing the activity of free lipase and lipase bound to cellulose nanofiber membranes over multiple uses.

### DETAILED DESCRIPTION OF THE INVENTION AND PREFERRED EMBODIMENTS

### I. Introduction

The present invention provides polymer-based nanofibers having multiple (i.e., a plurality of) nanopores and a biological material. The nanofibers described herein are typically two to three or more orders of magnitude smaller than fibers conventionally produced. The nanofibers also have a much higher surface area compared to other nanofibers due to the nanopores, which increase the effective surface area of the nanofiber by at least two or three or more orders of magnitude. The increased surface area provided by these nanopores increases the number of potential sites on the nanofiber which can interact with the biological material. Thus, materials having an increased biological activity can be produced using the nanofibers of the present invention. Moreover, due to the nanoscale sizes of these nanofibers, smaller quantities of the nanofibers are needed to achieve the targeted functions (e.g., applications that rely on materials' surface characteristics).

The nanofibers described herein can be incorporated into conventional textiles and other structures such as coatings, laminates, blends and additives. More particularly, the nanofibers described herein can conveniently be used for biological and chemical applications including, for example, in separation and filtration methodology (e.g., separation membranes), solid support catalysts (e.g., membranes for immobilizing biological materials), absorbent technology, pharmacological delivery systems, composite reinforcement (e.g., structural reinforcement of nonwoven and woven textiles), protective coatings (e.g., for protective clothing), recyclable catalysts, selective encapsulation, and, wound dressing materials, and scaffolds for cell and tissue growth (e.g., artificial blood vessels).

### II. Definitions

As used herein, the following terms have the meanings ascribed to them below unless otherwise specified.

The term “nanofiber” as used herein refers to continuous, ultra-thin fibers with diameters ranging from micrometers to nanometers. Typically, nanofibers have a diameter of about 50 nm to about 10,000 nm, about 50 nm to about 5,000 nm, about 100 nm to about 1,000 nm, or about 500 nm to about 1,000 nm. The nanofibers described herein may comprise a single type of polymer or multiple types of polymers. The polymers may be synthetic or naturally occurring. Nanofibers may be smooth or may have a plurality of nanopores (e.g., at least 2, 4, 10, 25, 50, 100, 200, 300, 400 or 500 nanopores). In some embodiments, the nanopores are on the surface of the nanofiber. In some embodiments, the nanopores traverse the diameter of the nanofiber. Nanofibers may further comprise a biological material. The biological material may be attached to the nanofiber (e.g., covalently or by adsorption) or may be incorporated into the nanofiber itself.

The term “nanopore” refers to a pore, hole, or depression on the surface of a fiber or nanofiber or a hole inside of a fiber or nanofiber. A nanopore typically has a diameter of about 5 nm to about 500 nm, about 10 nm to about 100 nm, about 25 nm to about 75 nm, about 10 run to about 50 nm, or about 5 nm to about 25 nm.

“Polymer,” as used herein, refers to a chemical compound or mixture of compounds formed by a chemical reaction in which single structural units combine to form a larger molecule comprising repeating structural units. Polymers include aqueous-soluble polymers, organic-soluble polymers, synthetic polymers, naturally occurring polymers, and crosslinked polymers. Aqueous-soluble polymers are polymers that dissolve in an aqueous solution such as, for example, water or a saline solution. Organic-soluble polymers are polymers that dissolve in organic solvents such as, for example, methanol, dimethylformamide, benzene, toluene, or acetone. Naturally occurring polymers are polymers derived from a biological organism such as, for example, polypeptides, cellulose, and carbohydrates. Any combination of the polymers can be crosslinked as described in detail below.

“Biological material” or “biomolecule,” as used herein, refers to any material from an organism, e.g., bacteria, yeast, reptiles, amphibians, birds, mammals and the like. Suitable biological materials include proteins and polypeptides, carbohydrates, nucleic acids, and the like. Exemplary proteins include enzymes (e.g., proteases, nucleases, kinases, and lipases), antibodies, growth factors, toxins, and the like. Biological materials may be naturally occurring (i.e., isolated or purified from the organism) or recombinant. Methods of recombinantly producing biological materials are well known in the art and are described in, e.g., Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

“Linker,” as used herein, refers to any molecule that can be used to attached a biological material to a nanofiber. Linkers attach the biological material to the nanofiber at an appropriate distance such that steric hindrance is minimized and the function of the biological material is maximized. Linkers may comprise a functional group that facilitates the covalent attachment of the biological materials to the nanofibers. Alternatively, the linkers may comprise a polar group on one end that facilitates the attachment of the biological material to the nanofiber. Typically, the linkers are polymers. Suitable linkers are well known in the art and include, for example, anionic electrolyte polymers (e.g., polyacrylic acid (PAA)), cationic electrolyte polymers (e.g., dimethylaminoethyl methacrylte (DMAEMA)), dipolar compounds (e.g., polyethylene glycol (PEG)), and non-ionic polymers (e.g., polyacrylamide (PAM)).

An “insoluble” material, as used herein, refers to a material (i.e., a nanofiber) that does not dissolve or lose its three dimensional structure when contacted with a solution, e.g., an aqueous or organic solution. In the context of a nanofiber, insoluble also refers to a nanofiber that retains its three-dimensional structure and any attached biological material when contacted with a solution. In some embodiments, a nanofiber can be rendered insoluble by crosslinking the polymer.

“Membrane,” as used herein, refers to a collection of fibers in sheet or layer form, typically generated by laying down individual fibers or nanofibers in a random fashion so that the fibers or nanofibers are interconnected or intertwined, e.g., in a matrix.

“Fabric,” as used herein, refers to a pliable material made up of fibers. Fabric is typically generated by weaving or knitting individual fibers or nanofibers so that the fibers or nanofibers are interconnected and form a pliable material.

“Porosity,” as used herein, refers to a measure of the permeability of the nanofibers to fluids. Porosity within the nanofibers can be measured by gas adsorption isotherm based on Lagmuir Brunauer, Emmett and Teller (BET) method. A porosity measurement of 0 indicates that a material is completely solid, i.e., impermeable to fluid. A porosity measurement of 1.0 indicates that a material is completely permeable to fluid. The nanofibers described herein typically have a porosity measurement of about 0.2 to about 0.95, about 0.3 to about 0.8, about 0.4 to about 0.75, or about 0.5 to about 0.6.

### III. Nanofibers and Compositions Comprising Nanofibers

In one embodiment, the present invention provides polymer-based nanofibers having a plurality of nanometer size pores (i.e., nanopores) and comprising a biological material. The biological material may be incorporated into the nanofibers or may be attached to the nanofibers as described in detail below. Advantageously, the nanofibers described herein have surface areas that are 3 to 4 orders of magnitude higher than conventional high specific surface area materials. Chemical reactions and polymer syntheses, polymer compositions and additives, and solvent systems are employed to form the nanofibers. Nanofibers can be made from a variety of materials including natural and synthetic polymers. Nanofiber surfaces can also be functionalized to allow attachment or linkage of the biological materials to the nanofibers. Typically, the ratio of protein to polymer is about 1:20 to about 20:1, about 1:10 to about 10:1, about 1:5 to about 5:1, about 1:4 to about 4:1. In some embodiments, the ratio of protein to polymer is about 1:1.

A. Polymers

The nanofibers described herein comprise at least one polymer. In some embodiments, the nanofibers comprise at least 2, at least 3, or more polymers. In a preferred embodiment, the nanofibers described herein comprise two polymers. Typically the first and second polymer are present in a ratio of about 1:20 to about 1:20 to about 20: 1, about 1:10 to about 10:1, about 1:5 to about 5:1, about 1:4 to about 4:1. In some embodiments the ratio of polymer to polymer is about 1:1.

A wide variety of polymers can be used for the nanofibers of the present invention. Suitable polymers include, but are not limited to, fibers from plants, polymers from animals, natural organic polymers, synthetic organic polymers and inorganic substances. In certain preferred embodiments, synthetic organic polymers are used. Suitable synthetic organic polymers include, e.g., organic-soluble polymers, aqueous-soluble polymers. Suitable organic-soluble polymers include, for example, polyacrylonitrile (PAN), polyamides, polyesters, polystyrene, polyvinyl chloride, polyvinyl acetate, cellulose derivatives, poly(acrylic acid) (PAA), polyethylene oxide (PEO), polypeptides, and combinations thereof. Suitable aqueous-soluble polymers include, for example, poly(acrylic acid) (PAA), poly(vinyl alcohol) (PVA), polyethylene oxide (PEO), polyvinyl pyrolidinone (PVP), poly(ethylene naphthalate), polyaniline, poly-L-lactide, and combinations thereof. One of skill in the art will appreciate that some polymers may be both organic-soluble and aqueous-soluble. In some embodiments, the polymeric materials suitable for use in the present invention include, but are not limited to, proteins (e.g., casein) and naturally occurring fibers from plants, such as cellulose, cotton, linen, hemp, jute, ramie, and derivatives thereof. Where the nanofibers comprise two polymers, the polymers may be the same (i.e., two synthetic organic polymers) or different (i.e., one synthetic organic polymer and one naturally occurring polymer).

B. Biological Materials

As described above, the nanofibers of the present invention comprise a biological material (i.e., a biomolecule). The biological materials may be incorporated into the nanfibers, as described in Example 3, below, or may be attached to the nanofibers after the fibers are prepared. If the biological materials are incorporated into the nanofibers, they can be incorporated before, during, and after generation of the nanofibers. The biological materials may be attached to the nanofibers using any means known in the art including, for example, via a linker (e.g., a molecule on the surface on the nanofiber comprising a functionalized group). The linkers attach the biological material to the nanofiber at an appropriate distance such that steric hindrance is minimized and the function of the biological material is maximized.

Biomolecules (i.e., biological materials) suitable for use in the nanofibers of the present invention, include, for example, a protein or polypeptide, an enzyme, an enzyme substrate, a hormone, an antibody, an antigen, a hapten, an avidin, a streptavidin, a carbohydrate, an oligosaccharide, a polysaccharide, a nucleic acid, or combinations thereof. Suitable proteins include, but are not limited to, integral membrane proteins; fibrous and structural proteins; intracellular proteins such as muscle proteins; extracellular proteins; enzymes; nucleic acid manipulation and regulation proteins such as polymerases, nucleases, and ligases, gyrases, topo-isomerases; DNA-binding proteins; response elements such as kinases, phosphatases, lipases; hydrolases such as nucleases and lipases; glycosidases; proteinases, and portions thereof. Preferred proteins include, for example, a lipase, a carbohydrolase, a DNAse, and a protease.

In one embodiment, a biological material is activated through an external stimulus. For example, if the nanofiber is a hydrogel as described below, raising the temperature may induce expansion of the hydrogel, thereby increasing the exposure of the biological material to its substrate. Similarly, contact with an appropriate solvent may expose portions of the nanofiber or alter the shape of the nanofiber so that any biological material attached or incorporated into the nanofiber is exposed. Thus, the external stimulus (i.e., a rise in temperature or contact with a solvent) has triggered or increased the activity of the biological material.

C. Compositions Comprising Nanofibers

In some embodiments, the invention provides membranes or fabrics comprising a matrix of the nanofibers described herein. Typically a membrane is nonwoven and a fabric is woven. In certain aspects, the membrane is generated by deposit of the nanofibers onto a surface such as, for example, paper, wood, metal. In some embodiments, deposit of the nanofibers is random. In other embodiments, the nanofibers are deposited as a three-dimensional scaffold (e.g., to support the growth of cells or tissues).

In certain aspects, the polymer is cross-linked to stabilize the naofibers, i.e., so that the nanofibers are insoluble in an aqueous or organic solution. Suitable cross-linking agents include, for example, isocyanates and derivatives thereof (e.g., 4,4′-methylenebis-(phenylisocyanate), methylene-diphenyl-di-isocyanate (MDI), toluene diisocyanate (TDI), and isophorone diisocyanate (IPDI)), aziridines and derivatives thereof, or epoxies and derivatives thereof. Those of skill in the art will appreciate that there are multiple other agents suitable for use in the present invention.

In one embodiment, the nanofibers are immobilized on a solid support or a solid phase. For example, the nanofibers can be further attached to wells, raised regions, dimples, pins, trenches, rods, pins, inner or outer walls of cylinders, and the like. Other suitable support materials include, but are not limited to, agarose, polyacrylamide, polystyrene, polyacrylate, hydroxethylmethacrylate, polyamide, polyethylene, polyethyleneoxy, or copolymers and grafts of such. Other embodiments of solid supports include small particles, non-porous surfaces, and addressable arrays.

### IV. Methods of Making

The nanofibers described herein can be made using any method known to those of skill in the art. In a preferred embodiment, the nanofibers are made via electrospinning. Nanopores are generated in the nanofibers before, during, or after electrospinning by one or more of the following methods: synthesis of interpenetrating networks, polymerization, copolymerization, crosslinking, differential etching/dissolution.

A. Electrospinning

Electrospinning (ES) is a process capable of producing ultra thin polymer fibers with diameters ranging from microns to nanometers. The organic soluble polymers can be polyacrylonitrile (PAN), polyamides, polyesters, polystyrene, polyvinyl chloride, polyvinyl acetate, cellulose derivatives, poly(acrylic acid) (PAA), polyethylene oxide (PEO) and proteins. The aqueous soluble polymers can include poly(acrylic acid) (PAA), poly(vinyl alcohol) (PVA), polyethylene oxide (PEO), polyvinyl pyrolidinone (PVP), for the preparation of homopolymer solutions or mixtures.

Typically, a solution comprising at least one polymer is dissolved in a solvent and placed in a glass pipet tube sealed at one end with a small opening in a necked down portion at the other end. A high voltage potential (>50 kv) is then applied between the polymer solution and a collector near the open end of the pipet. This process can produce nanofibers with diameters of about 50 nm to about 2,000 nm, although diameters of about 10 nm to about 10,000 nm can also be produced. In certain aspects, an electrospinning apparatus, comprising a high voltage power supply (e.g., ES30P/100, Gamma High Voltage Research Inc.), a polymer solution reservoir, and a target or collector is used. A polymer solution is fed through a glass tube with capillary opening of approximately 1 mm in diameter. A metal (e.g., stainless steel or copper) pin immersed in the solution serves as the electrode and is connected to a high voltage source. (see, e.g., Liu and Hsieh, J. Polymer Sci. and Polymer Physics 40:2119-2129 (2002); Liu and Hsieh, J. Polymer Sci. and Polymer Physics (41):953-964 (2003); and Xie and Hsieh, J. Mat. Sci. 38:2125-2133 (2003)). With the adjustment of an electrical field, the electrostatic force overcomes the surface tension of the drop, ejecting the jet toward the target. Changes of the polymer jets cause “splaying” or longitudinal splitting of the jet into finer streams. Upon evaporation of the solvent, dried fibers are collected on the counter electrode in a fibrous web. By controlling the solvent systems and solution properties, fibers with diameters of 100-500 nm can be produced from several different polymers.

One of skill in the art will appreciate that various parameters can be adjusted to generate electrospun fibers with a desired morphology. For example, solution viscosity, net charge density, surface tension, accelerating voltage, solution concentration, polymer concentration, deposition distance, applied field strength, and deposition time can all be adjusted to regulate fiber morphology (see, Fong et al., Polymer, 40:4585 4592 (1999); Deitzel et al., supra, and Buchko et al., supra). Typically, fiber diameters generally decrease with decreasing polymer concentration (i.e., viscosity) and with increasing applied field strength; dilute polymer solutions often lead to the formation of beaded or irregular fibers; and short deposition distances may produce film composed of flat or merged fibers.

B. Generation of Nanopores

Several approaches are used to create nanopores in the nanofibers described herein. One of skill in the art will appreciate that chemical strategies (i.e., methods) and physical strategies (i.e., methods) can be used to generate the nanopores. The chemical and physical methods can be used before, during, or after generation of the nanofibers. Suitable chemical strategies include, for example, in-situ polymerization and copolymerization, synthesis of interpenetrating networks, and chemical crosslinking, all of which can conveniently be used to generate nanopores. Suitable physical strategies include, for example, the use of additives, phase separation techniques and selective etching/dissolution techniques. Each of these strategies can be used alone or in combination with other strategies to produce nanopores. Nanometer size pores (i.e., nanopores) can be formed inside the nanofibers and on the surface of the nanofibers.

1. In-situ Polymerization and Copolymerization of Interpenetrating Polymer Networks.

In situ polymerization and copolymerization can be used to generate nanopores in the nanofibers described herein. Expandable three-dimensional polymer networks can be made into fibrous structures by polymerization and formation of interpenetrating polymer networks (IPNs). Typically, one polymer is used as the carrier for the monomers and other components in the fiber formation process. The copolymerization and polymerization reactions occur either during or immediately following the formation of the nanofibers.

Polymerization to generate nanopores on the surface of nanofibers as well as within the nanofibers can be controlled by adjusting different components of the polymerization reactions (e.g., the polymerization initiator, solvents, and cross-linkers). For example, free radical polymerization of styrene can be initiated by selected initiators which generate gaseous products upon thermal decomposition. Alternatively, differential evaporation or deposition of mixed solvents or non-solvents can produce three-dimensional polymer networks, upon swelling in a particular solvent. Finally, cross-linkers (e.g., divinylbenzene (DVB)) can be added to the reaction system before the polymerization. Before the formation of a complete swollen-gel, the mixture will be electrospun to create a three-dimensional electrospun membrane. The solution will easily tend to gel with increasing concentrations of the cross-linker. Other additives, such as, for example, blowing agents and low boiling solvents can be added to control the nanopore morphology of the fibers.

Formation of nanofibers can be adjusted by controlling the viscosity of the polymerizing solutions. Additional factors such as molecular weight and crosslinking effects directly influence the nanofiber properties as well as efficiency of nanofiber formation. The monomer can be present at up to about 50% and the crosslinker can be present at up to about 10%.

Copolymerization of at least two compatible but structurally different monomers can be used to generate a polymeric network structure of nanofibers comprising nanopores in the nanofibers. For example, styrene can copolymerized with other vinyl monomers in the presence of a crosslinker. In the case of maleic anhydride (MA), copolymerizing with styrene (S) in the presence of a thermal initiator (e.g., 2,2′-Azobisisobutyronitrile (AIBN)) produces a 1:1 alternating PSMA copolymer product. Varying molecular weights be can achieved by controlling the monomer/initiator (M/I) ratios. The PSMA can be dissolved in a solvent (e.g., dimethylformamide (DMF) at about 10% to about 30% and electrospun into nanofibers. Hydrolysis of PSMA converts maleic anhydride to hydrophilic carboxylic acid, introducing hydrophilicity and widening the solvent options for fiber formation. The PSMA fibers have diameters of at least 50 nm and above. After hydrolysis, the fibers are swollen and have an increased surface area and diameter. To facilitate attachment of biological materials to the nanofibers, functionalized surfaces can be created on the nanofibers. For example, the benzene pendant group of the styrene can be functionalized as depicted in Scheme I below. Post-fiber formation hydrolysis of the MA increases the hydrophilicity of these fibers and produces surface COOH groups which can be modified into other functional groups.

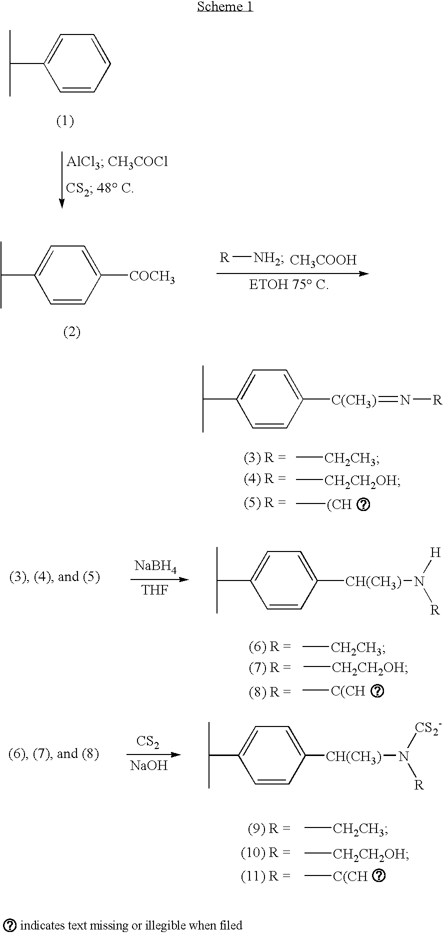

2. Chemical Crosslinking

In some embodiments, nanopores can be generated by inter-polymer crosslinking. Chemical crosslinking reactions target various polymer side groups or added compounds with either bi- or multi-functional reactive groups (i.e., reactive pairs or reactive sets). These reactions lead to the formation of 3-dimensional polymer networks. Suitable reactive pairs include, for example, carboxyl and hydroxyl, amine and carboxyl, aldehyde and aldehyde, aldehyde and carboxyl. One of skill in the art will appreciate that the reactions described below may be accelerated by the use of thermal (e.g., heat) or chemical catalysts.

In some embodiments, two polymers are crosslinked. Two polymers having complementary reactive groups are mixed and electrospun into nanofibers having 100 nm or larger diameters. In an exemplary embodiment, nanofibers are made by electrospinning a mixture of PAA and PVA polymers (COOH:OH ratios between 5 to 0.2). These nanofibers can be thermally crosslinked; esterification between the carboxylic acid groups of PAA and the hydroxyl groups of PVA occurs at elevated temperatures. These crosslinked nanofibers behave like hydrogel gels and swell about two or three times to about two or three thousand times their dry mass. The pores in between 3-D gel structures are typically macropores (>50 nm) and mesopores (>10 nm). These nanoporous nanofibers are stable in aqueous solutions (e.g., water and buffers of varying pH) and organic solvents. Furthermore, nanofibers are responsive to multiple stimuli, i.e., the fibers are sensitive to environmental changes including, pH, electric fields, and ionic strength because of the dissociation of carboxylic acid groups of PAA. Significant increase in swelling of these fibers occurs around a pH of 4.7, near the pKa of the carboxyl acid groups. The application of electric field will further increase the swelling of these fibers.

In some embodiments, three polymers are crosslinked. For example, PNI/PAAm/PAA fibers can be crosslinked by mixing a catalyst (e.g., Na₂HPO₄ or PVA) with the ES solution and curing the fiber at 140° C. or higher temperatures. Na₂HPO₄ facilitates the crosslinking by lowering the activation energy of the reaction, while PVA improves the compatibility of the polymers, thereby enhancing the crosslinking reaction more significantly. The nanofibers behave like hydrogels and their swelling behavior in liquids can be controlled by adjusting the relative proportion of the polymers used. These nanofibers are insoluble in solvents that would otherwise dissolve the individual polymer components prior to electrospinning. Such hydrogel fibers are responsive to multiple stimuli, e.g., they exhibit sensitivity to pH changes (e.g., between pH 4 and 5) and temperature changes (e.g., between 30° C. and 70° C.).

In some embodiments, bi- or multi-functional compounds can be used to crosslink the polymers. For example, bi- or multi-functional compounds bearing aldehyde, acylcholoride, carboxylic or amine end groups can be used during or after electrospinning to generate chemical crosslinks between carboxyl group carrying polymer chains (e.g., PAA, carboxymethyl cellulose). Suitable bi- or multifunctional compounds include, e.g., di-carbodiimide (EDC), polyol diacylchloride, polyol multi-acylchloride, dialdehyde, etc. For instance, 1 -ethyl-3-(3-dimethylaminopropyl) carbodiimide (EDC) is used to react the COOH groups in the fibers and primary amine of another compounds or polymer in the presence of N-hydroxysulfosuccinimide (NHSS).

3. Use of Additives

Additives can also be used to generated nanopores in the nanofibers described herein. Additives useful for generating nanopores exhibit significantly different chemical and physical properties from the polymer material such that they aggregate into small domains within the polymer fibers. Additives that are soluble or dispersible in the solvent used to dissolve the polymer can conveniently be added to the polymer solution and the polymer/additive solutions can be elelctrospun into continuous fibers. Typically, the additives are added into the polymer solution before electrospinning, and removed from the polymer fibers during or after electrospinning. Suitable additives include, but are not limited to, liquids, gases, crystals and crystallizable compounds, nano-particles, and combinations thereof.

a) Liquids

In a preferred embodiment, volatile liquids are used to generate nanopores. Suitable volatile liquids include, for example, ethers, pentane, methylene chloride, alcohols, acetone, tetra-hydrofuran (THF), chloroform, carbon tetrachloride, and hexane. In some embodiments, the volatile liquids (i.e., liquid additives) are solvents in which the polymer material is soluble; in other embodiments, the volatile liquid is a solvent in which the polymer material is insoluble. The volatile liquids may be completely or partially miscible with the polymer solvent. Typically, the liquid additives are added to the polymer solutions in varying amounts. The more evaporative liquid additive either escapes from the polymer jet more quickly than the solvents with or without an aid such as heat during electrospinning. As a result, pores are created on the fiber surface or in the fiber.

b) Gases

In some embodiments, additives that decompose completely far below the decomposition temperature of the polymer release gas products, such as oxygen, during the solidification of the fibers, thereby forming pores. One such compound is potassium persulfate (K₂S₂O₈) which can be added into the polymer solution before the electrospinning. Such a heating process may serve to crosslink as well as to release gaseous products. As a result, pores will be created on the nanofiber surface and/or within the nanofibers.

c) Crystals or Crystallizable Compounds:

In some embodiments, crystals or crystallizable compounds are used as additives to generate nanopores in the nanofibers described herein. The crystals or crystallizable compounds are dissolved in polymer solutions prior to electrospinning and removed after electrospinning by contacting the nanofiber with a solvent that is an effective solvent of the crystal or crystallizable compound, but a poor solvent of the polmer matrix. The resulting porous nanofibers will have pores on their surfaces and internally. In an exemplary embodiment, a crystallizable small molecule, such as β-cyclodextrin, is dissolved in a polymer solution prior to electrospinning. After electrospinning, the β-cyclodextrin crystals are selectively removed from the fibers by contacting the nanofibers with a solvent such as pyridine. One of skill in the art will appreciate that a variety of crystals, crystallizable compounds, and solvents can be used to generate the porous nanofibers.

d) Nano-Particles

In some embodiments, nano-particles are used as additives to generate nanopores in the nanofibers described herein. The nano-particles can be added into polymer solutions prior to electrospinning, with or without the help of surfactants or dispersing agents. Suitable nano-particles include, for example, oxides of metals, silver, carbonaceous particulates, carbon nano-tubes, and combinations thereof.

4. Phase Separation and Differential Solubility

In some embodiments, phase separation and differential solubility can be used to generate nanopores in the nanofibers described herein. In this embodiment, at least two polymers are electrospun to generate nanofibers. The polymers remain in different phases even after they are electrospun and they are differentially soluble. After the nanofibers are prepared, selective etching and dissolution of portions of the nanofibers can be used to generate nanopores after electrospinning. To selectively etch portions of the nanofibers, the nanofibers are contacted with a solvent which selectively removes one of the polymer or polymers from the nanofiber. One of skill in the art will appreciate that the duration of contact and the concentration of solvent may be adjusted to control the morphology (i.e., size and shape) of the nanopores.

In a preferred embodiment, two polymers are used for the nanofiber: one polymer that is organic-soluble (i.e., polymer A) and one polymer that is both organic-soluble and water-soluble (i.e., polymer B). The polymers remain in different phases even after they are electrospun into a single nanofiber. The polymers may be present in a ration of about 1:20 to about 20:1, about 1:10 to about 10:1, about 1:5 to about 5:1, about 1:4 to about 4:1, or about 1:1. After electrospinning, the nanofibers are contacted with an aqueous solvent, thus generating nanopores in portions of the nanofibers comprising the water-soluble polymer. Alternatively, the nanofibers are contacted with an organic solvent, thus generating nanopores in portions of the nanofibers comprising the organic-soluble polymer.

In another preferred embodiment, two polymers are used for the nanofiber, both of which are organic soluble. The polymers may be present in a ratio of about 1:20 to about 20:1, about 1:10 to about 10:1, about 1:5 to about 5:1, about 1:4 to about 4:1 or about 1:1. After electrospinning, the nanofibers are contacted with an organic solvent that is effective against only one of the polymers, thus generating nanopores in portions of the nanofibers. In an exemplary embodiment, polymethyl methacrylate (PMMA) or polystyrene (PS) can be dissolved with PAN in DMF and electrospun into nanofibers. Chloroform, a good solvent for both PMMA and PS but a non-solvent for PAN can be used to remove PMMA or PS from the fibers, creating nanoporosity in the PAN fibers.

C. Linkage of Biological Material to Nanofibers

In some embodiments, a biological material is attached to the nanofiber after electrospinning. Several chemical approaches have been exploited to activate fiber surfaces and to incorporate enzyme molecules as well as other proteins. Suitable linkers for attaching the biological material to the nanofiber may comprise a functional group (e.g., an ester, an amide, a carbamate, a carbonate, a thioester, and a thiocarbamate) that facilitates the covalent attachment of the biological materials to the nanofibers. Alternatively, the linkers may comprise a polar group on one end that facilitates the attachment of the biological material to the nanofiber. Typically, the linkers are polymers. Suitable linkers include, for example, anionic electrolyte polymers (e.g., polyacrylic acid (PAA)), cationic electrolyte polymers (e.g., dimethylaminoethyl methacrylate (DMAEMA)), dipolar compounds (e.g., polyethylene glycol (PEG)), and non-ionic polymers (e.g., polyacrylamide (PAM)).

Various linking systems employing chemical bonding mechanisms to bind enzymes and other proteins on fibrous materials have been successfully developed. As such, in another aspect, the present invention provides methods of activating fibers such as polymer fibers, and coupling the activated fibers with proteins (e.g., enzymes). The following reaction schemes merely exemplify and do not limit the claimed invention. Those of skill in the art will recognize variations and alternatives.

Scheme II below exemplifies the conversion of a carboxylic group to an acyl chloride of a linker (A) and thereafter, the reaction with a hydroxyl group containing fiber (B):

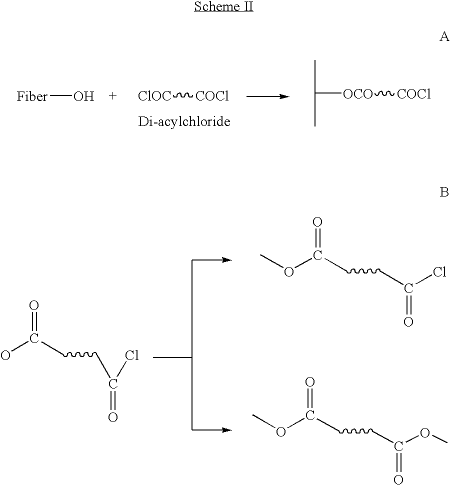

The B products can be either functional or a crosslinked structure. The functional product can be readily optimized by controlling the molar ratio of the reactants. The nature of the linker depends on its structure and can be either hydrophilic or hydrophobic.

The surface modified fibers having, for example, either acid or acylchloride end groups, can then be reacted with for example, the amine of the ε-aminolysine in an enzyme to form an amide bond, as shown in Scheme II.

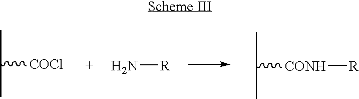

In a preferred method of attaching a biomolecule such as an enzyme to a fiber, a reactive group reacts with for example, a thiol, a hydroxyl, a carboxyl, or an amino group on a biomolecule, forming an attachment between the fiber and the biomolecule. In certain aspects, a fiber and a biomolecule form a covalent bond between the fiber and the biomolecule. The bond is for example, an amide, a secondary or tertiary amine, a carbamate, an ester, an ether, an oxime, a phosphate ester, a sulfonamide, a thioether, a thiourea, or a urea.

When linking a fiber having a carboxylic acid, with an amine-containing biomolecule, the fiber carboxylic acid can first be converted to a more reactive form using an activating reagent, to form for example, a N-hydroxy succinimide (NHS) ester, a mixed anhydride or acyl chloride as above. The amine-containing biomolecule is treated with the resulting activated acid to form an amide linkage. Typically, this reaction is carried out in aqueous buffer with an optional cosolvent.

Similarly, the attachment of an isothiocyanate containing fiber is analogous to the procedure above, but no activation step is required. The amine-containing biomolecule is treated directly with the NCS fiber to form a thiourea linkage. The efficiency and yield of this enzyme coupling reaction is quite good. In certain instances, it is possible to increase yield by optimizing parameters such as for example, temperature, pH, and time. The effects of the length and structure of optional linkers have also been investigated.

In other aspects, methods and processes of the present invention employ chemical strategies such as linkers or spacers with functional end groups that can further react with enzyme molecules. For example, the present methods include 1) incorporating enzymes and proteins in fibers by activating fiber surfaces and introducing chemical linkers that can react with enzyme proteins, and thereafter evaluating enzyme activities. Further, the biomolecules can be crosslinked and/or multi-functional reagents and surface reactive functional groups can be used to yield strong bonds and stable enzyme solid complexes.

In certain aspects, functional groups on enzyme proteins that are utilized for the covalent binding, include, for example, the N-terminus or the ε-aminolysine of an enzyme. Work on reactions of enzymes has shown that physicochemical properties of enzymes modified at the ε-aminolysine are only slightly altered, even in markedly modified proteins, indicating that ε-aminolysine residues are non-essential for catalytic activity. To react with an ε-aminolysine, functional groups including —OH, carboxylic —COOH, and aldehyde —C(O)H groups can be used. In certain instances, these primary functional groups are introduced on to the fiber. In other aspects, the present invention provides methods including the selection of an enzyme; the modification and activation of fiber surfaces which are reactive toward the ε-aminolysine of the enzyme, the development of an assay to evaluate enzyme activities, and the applications of these toward binding enzyme on selected fibrous materials.

In some embodiments, the biological material is attached to the polymer prior to formation of the nanofibers.

### V. Uses

The ultra-high surface area fibrous materials of the present invention (i.e., nanoporous nanofibers) can conveniently be used in a variety of applications including, for example, chemically and biologically protective coatings, recyclable catalysts, reactive and smart materials, and targeted separation membranes. In certain instances, the methods and compositions of the present invention are useful to generate novel fibrous supports for encapsulation (i.e., when biological materials are incorporated into the nanofibers themselves) and or immobilization of biomolecules (i.e., when the biological materials are attached to the nanofibers after they are produced). In certain aspects, the nanofibers can be used to make textiles, such as protective clothing for biological warfare. In certain instances, the present invention provides wipes, sponges, and clothing for the decontamination of equipment and personnel upon exposure to toxic chemicals and biological agents. In addition, the compositions and methods can be used for solid support catalysts, membrane supported smart devices, sensors and the like.

The nanofibers described herein can be incorporated into conventional textiles and other structures such as coatings, laminates, blends and additives. More particularly, the nanofibers described herein can conveniently be used for biological and chemical applications including, for example, in separation and filtration methodology (e.g., separation membranes), solid support catalysts (e.g., membranes for immobilizing biological materials), absorbent technology, pharmacological delivery systems, composite reinforcement (e.g., structural reinforcement of nonwoven and woven textiles), protective coatings (e.g., for protective clothing), recyclable catalysts, selective encapsulation, and, wound dressing materials, and scaffolds for cell and tissue growth (e.g., artificial blood vessels).

As described above, the surface chemistry principles for creating reactive fibrous materials are capable of binding with biomolecules. In some embodiments, the biomolecules such as enzymes are recovered and reused. In other embodiments, the nanofibers, membranes comprising the nanofibers, or fabric comprising the nanofibers are reused.

### EXAMPLES

### Example 1

Generation of Nanoporous Nanofibers by Phase Separation and Differential Solubility

Nanopores were generated in nanofibers comprising two polymers: polyacrylonitrile (PAN) and polyethylene oxide (PEO) using phase separation and differential solubility. Both PAN and PEO are organic-soluble, PEO is also water-soluble. Organic solutions comprising PAN and PEO, with varying PEO concentrations (15%, 20%, and 50%) were electrospun on an electrospinning apparatus comprising a high voltage power supply (ES30P/100, Gamma High Voltage Research Inc.), capable of processing polymer solutions, to yield ultra-fine cylindrical nanofibers of smooth surfaces and homogeneous dimensions. (Viscosities of 8% polymer solutions are 31.2 cP and 2.7 cP, for PAN and PEO, respectively). The nanofiber diameters ranged from about 500 nm to about 100 nm, with increasing amounts of PEO. These fibers have 200 times the specific surface area of conventional fibers. The distinct thermal behaviors of the individual polymers were detected by differential scanning calorimetry (DSC) to confirm clear phase separation of the two polymers, in the form of cast films as well as in the electrospun nanofibers.

Removal of PEO results in mass losses of 17%, 31% and 49% from membranes comprising the nanofibers, percentages very close to the masses of PEO in the original nanofibers. The nanofiber surfaces also become rough. Membranes comprising the nanofibers remain fibrous upon prolonged exposure to aqueous media. Fourier Transform Infrared Spectroscopy (FTIR) confirmed that differential solubility is an efficient means to remove the phase-separated domains of PEO.

Experiments using a liquid inclusion method show that inter-fiber porosity can be controlled between 0.3 to 0.95. Porosity within the fibers can be measured by gas adsorption isotherm based on Lagmuir Brunauer, Emmett and Teller (BET) method. Intra-fiber porosity has shown to also significantly increase with the removal of PEO. BET measurements of the nanofibers showed that removal of PEO significantly increased the intra-fiber pore volume (50% from 0.26 cc/g to 0.37 cc/g) and specific surface area (three-fold from 18.9 to 49.7 m²/g) of the fibers. The diameters of these nanopores range from 8 to 60 nm, depending upon the particular methods and substrates used.

These results indicate that (a) electrospinning of solvent-compatible yet phase-separated polymers generates uniform fibers with high efficiency; (b) fiber sizes and inter-fiber porosity can be easily controlled by polymer compositions and solution properties; and (c) differential solubility removes phase-separated domains and generates nanoporosity.

### Example 2

Generation of Cellulose Membranes with Attached Lipase

Preparation of cellulose nanofibers: 1.5% cellulose acetate (CA) was prepared by dissolvig CA in 1:2 mass ratio of N, N-dimethylacetamide (DMAc):acetone mixture. The CA solution was electrospun at 10 KV and collected onto a grounded aluminum collector at 7 inches. Deacetylation was carried out in 0.05 M NaOH for 24 hours at room temperature. CA membranes were rinsed with dH₂O to stop the reaction and dried for 10 hours at 80° C. under vacuum.

Lipase: Lipase EC 3.1.1.3 from Candia rugosawas used in this study. Lipase activity was assayed using olive oil as described below.

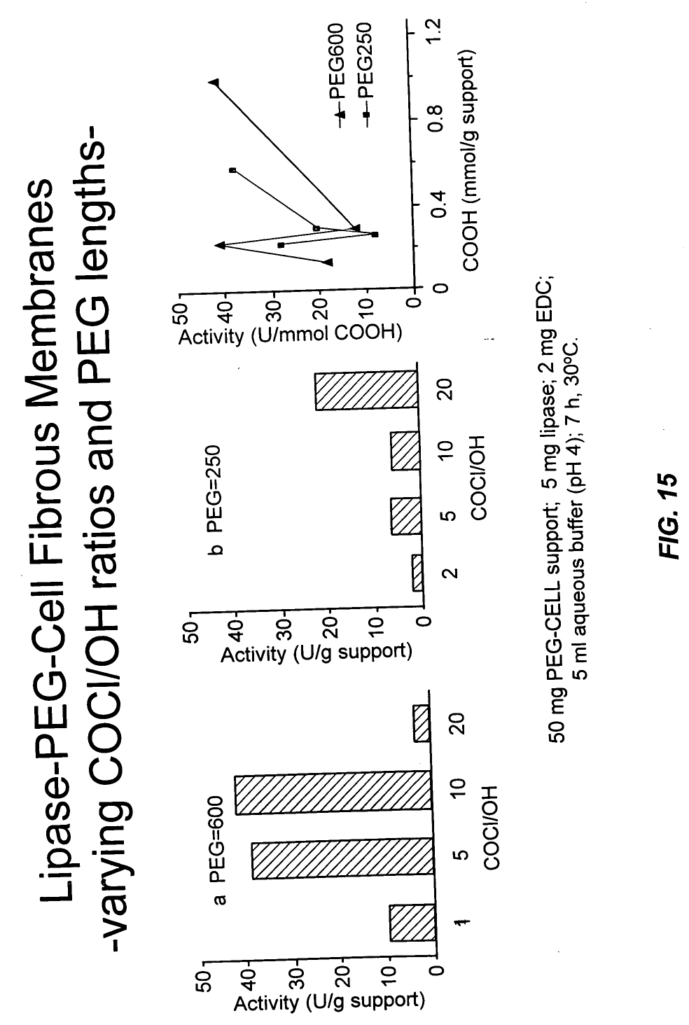

Immobilization: Lipase was immobilized onto the cellulose membrane by either adsorption or covalent binding. For adsorption, PAA was grafted onto the cellulose nanofibers via ceric ion initiation. The carboxylic acid concentration was controlled by varying AA and/or ceric ion concentrations. Fibers with 3.6 mM COOH per g cellulose were used for the enzyme adsorption and assays. PAA activated cellulose fibers were immersed in 1.0 ml/ml lipase solution for 24 hours at room temperature. The fibers were then rinsed in neutral (pH 7.0) buffer and dH₂O and dried for 12 hours at room temperature under vacuum. For covalent binding, PEG-diacylchloride (COCl-PEG-COCl) was attached to cellulose nanofibers via ester bond formation between the COCl of the PEG-diacylcloride and the OH on the cellulose. The quantities of COCl and COOH can be optimized by varying the COCl/OH molar ratio and PEG chain length (see, e.g., FIG. 15). The reacted nanofibers comprised 1.0 mM PEG per g of cellulose. Lipase was covalently bonded with the PEG-attached cellulose nanofibers in acidic (pH 4.0) buffer for 7 hours in the presence of a cabodiimide (EDC) coupling agent. The nanofibers were then rinsed in buffers of increasing pH: 4.0 to 7.0 to 10.0, then dried for 12 hours at room temperature under vacuum.

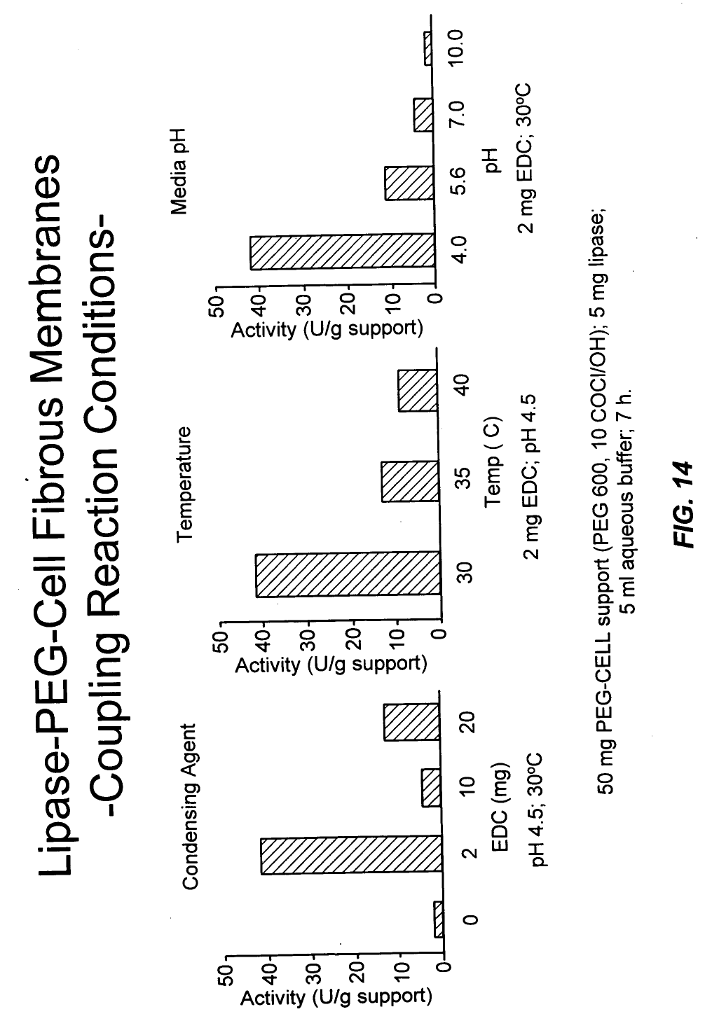

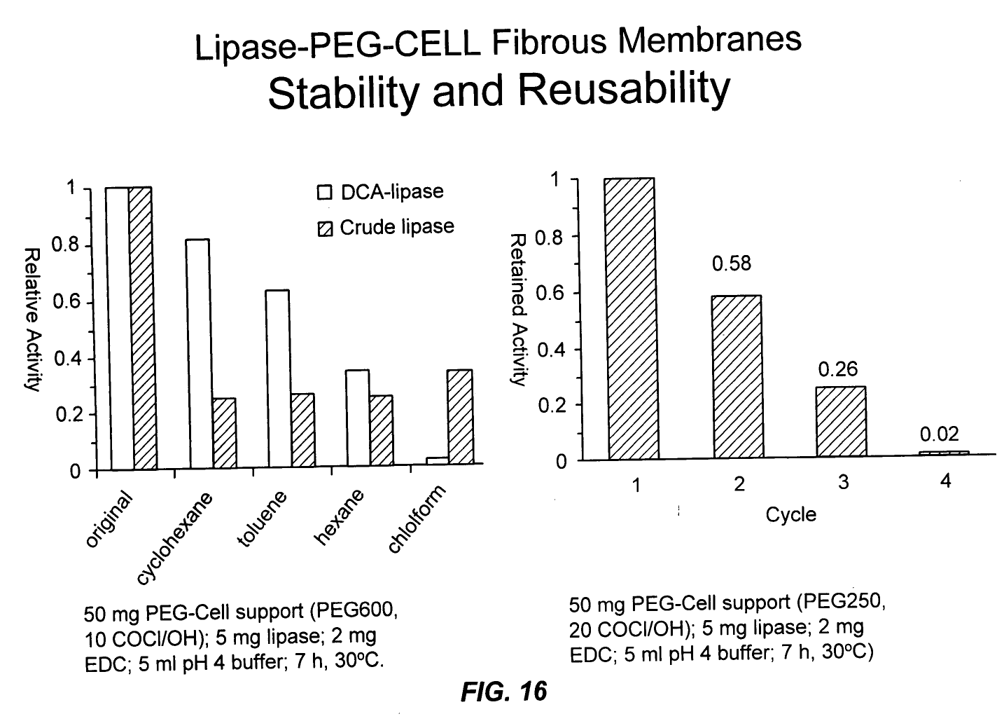

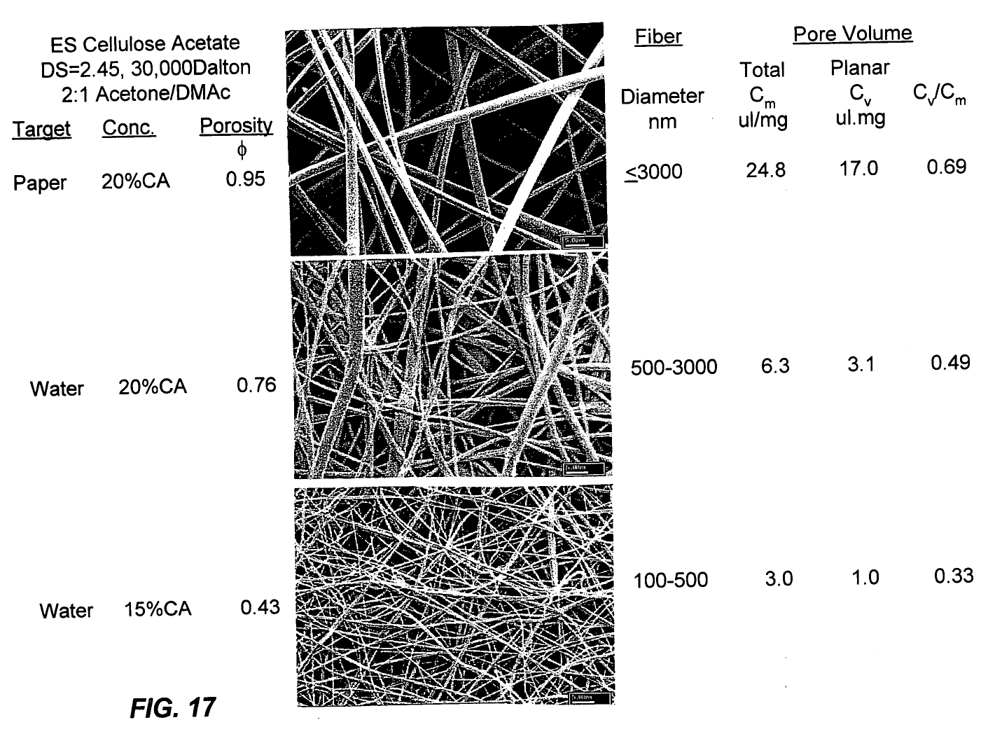

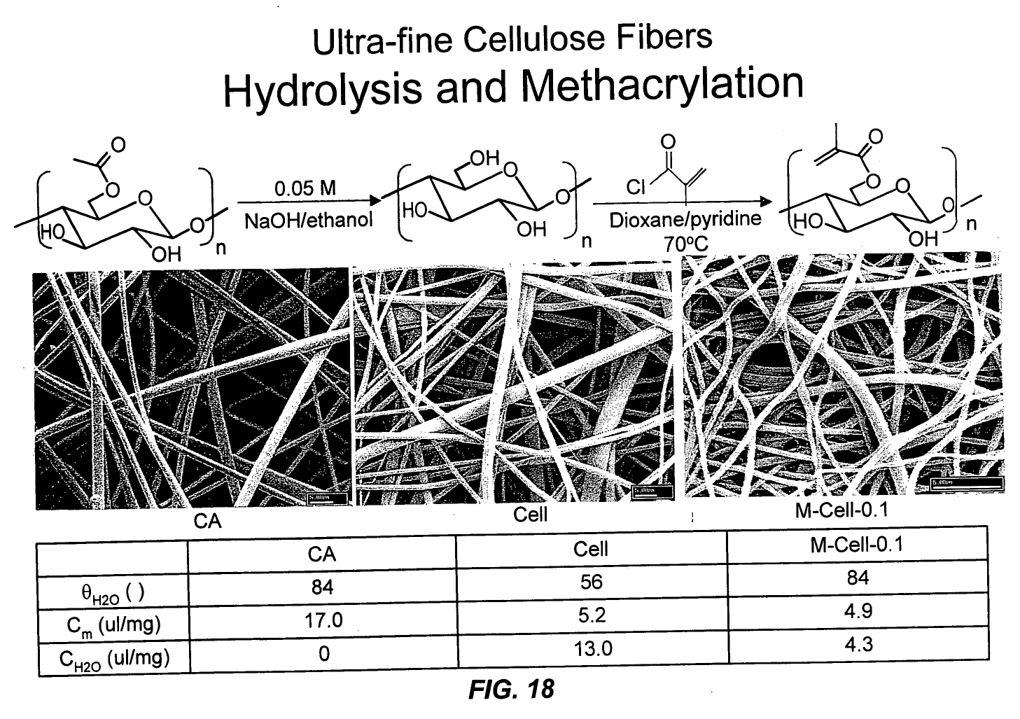

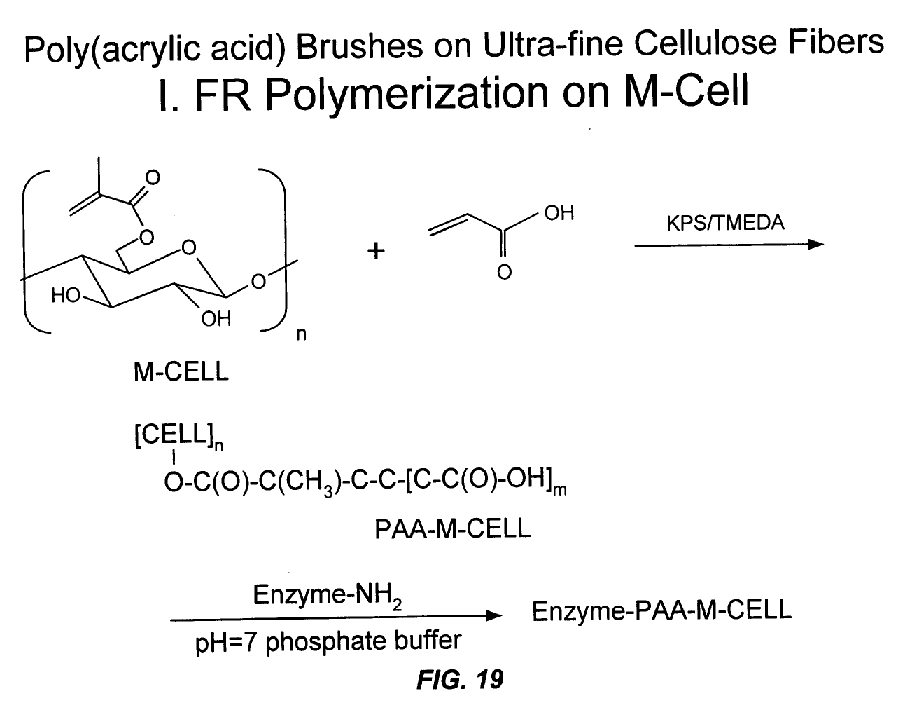

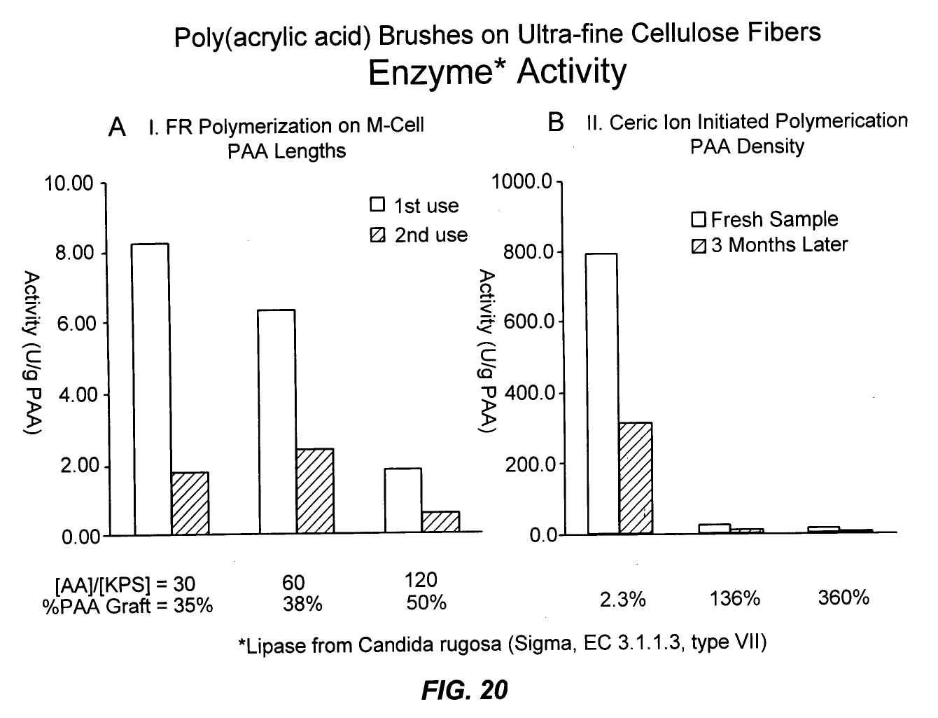

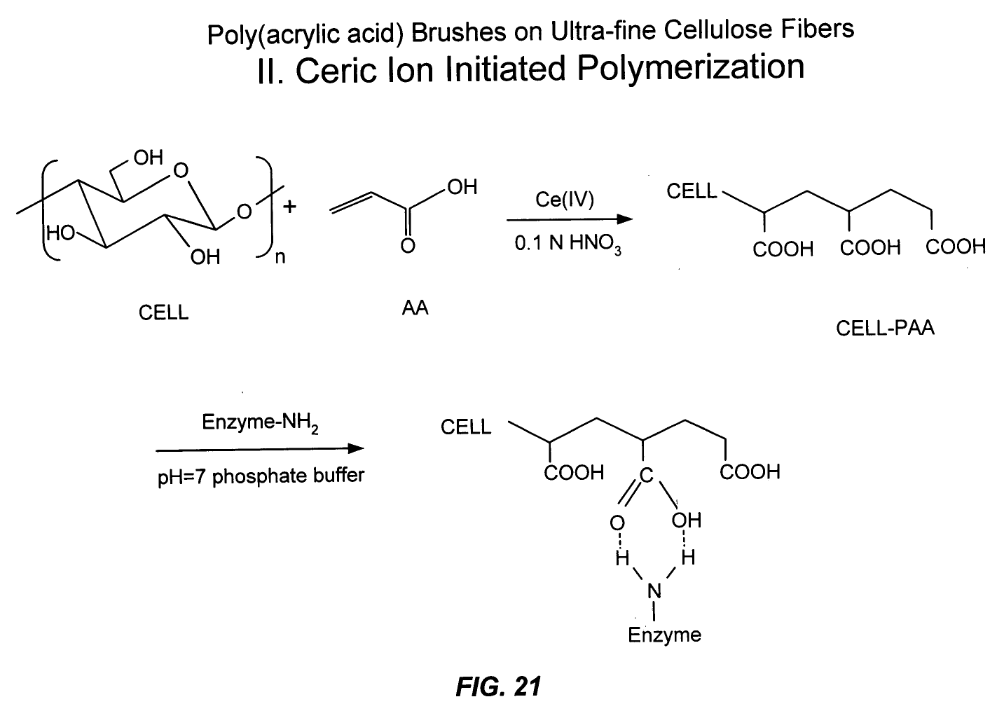

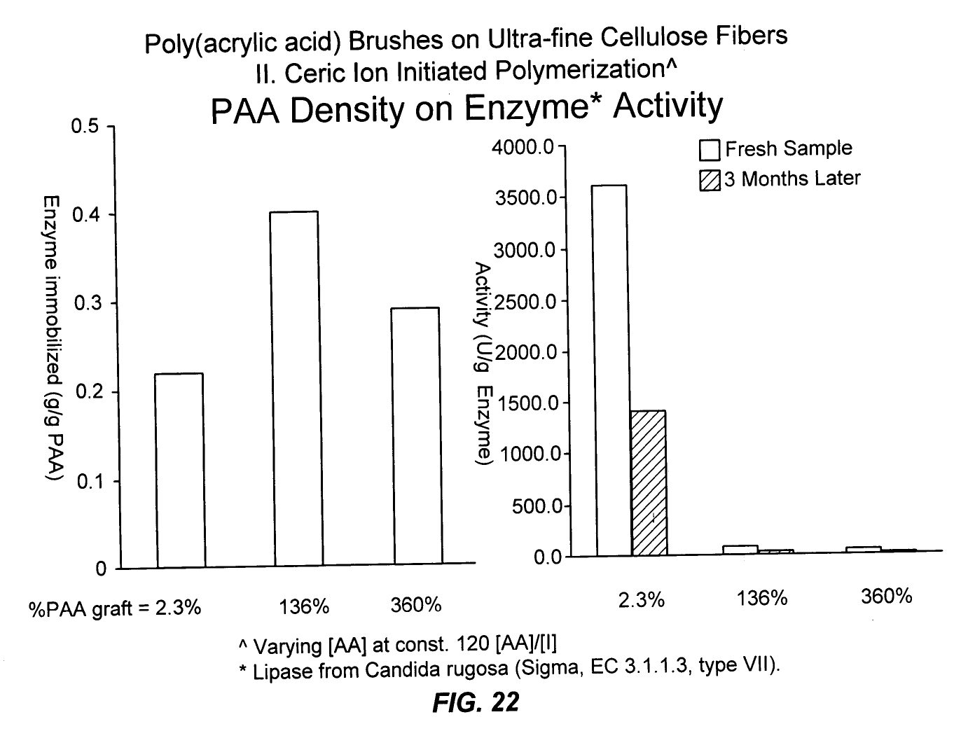

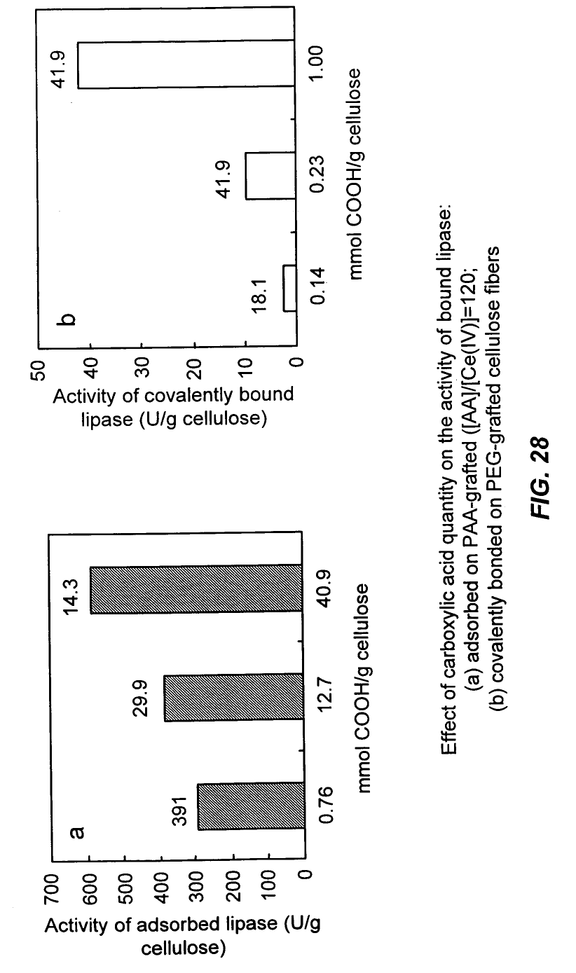

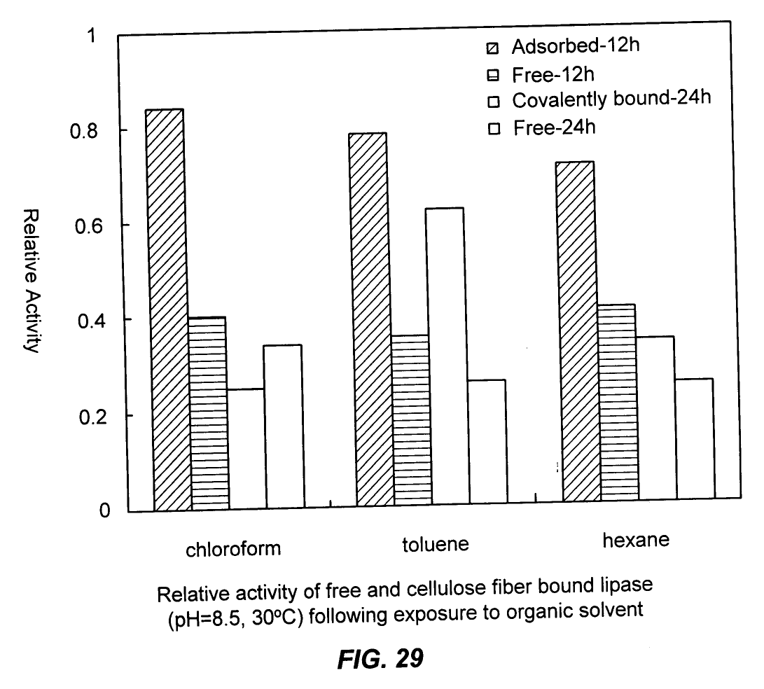

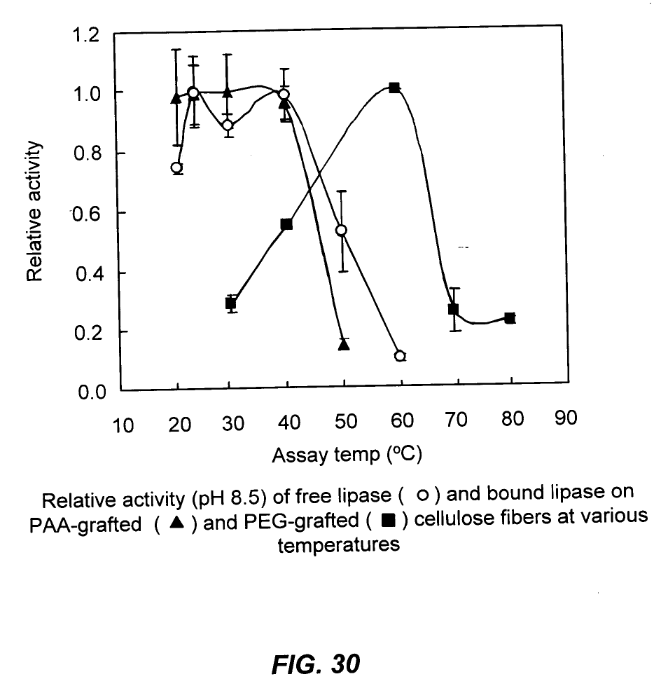

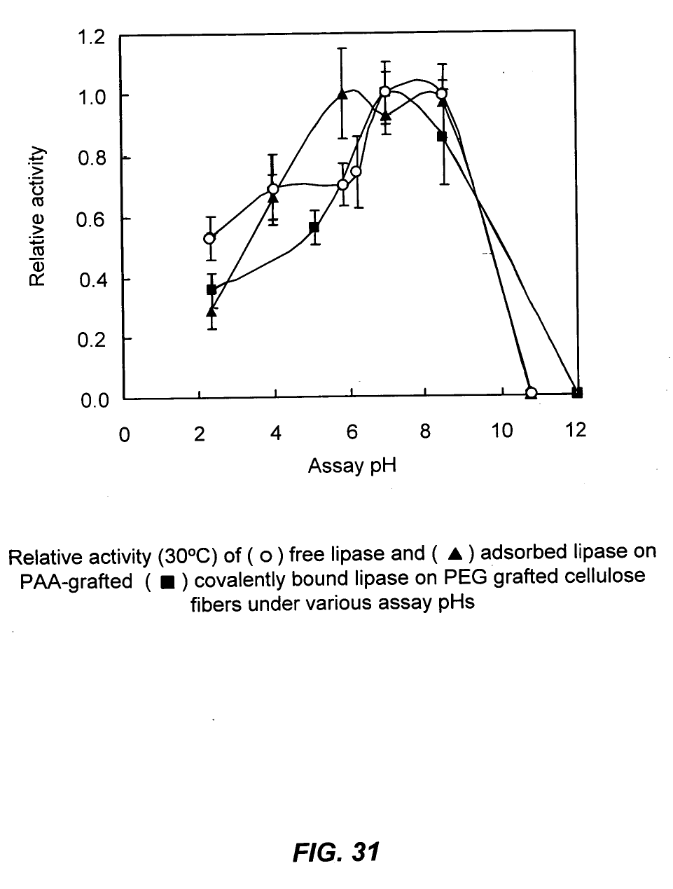

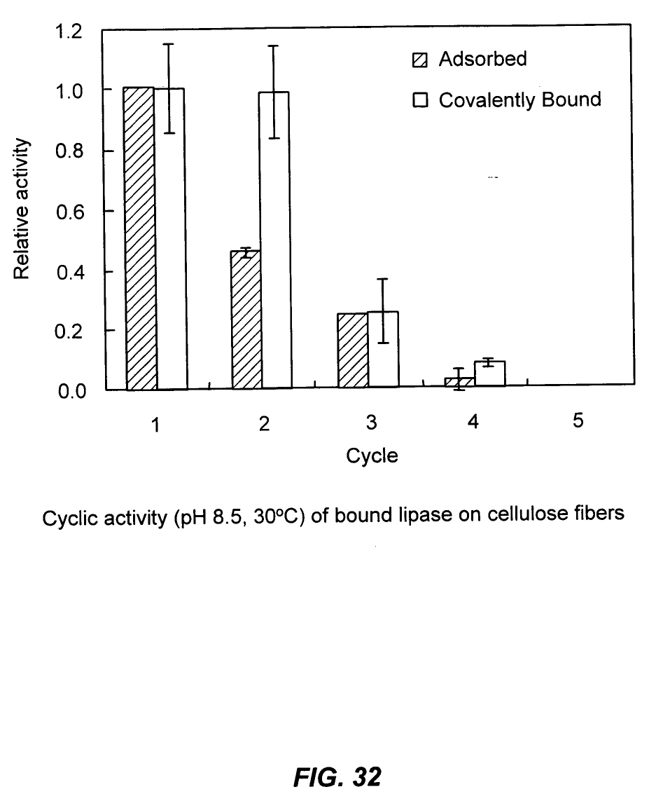

Results: The amount of COOH introduced by PAA grafting onto the nanofibers ranged from 0.76-40.9 mM/g cellulose. The amount of COOH introduced by PEG grafting onto the nanofibers ranged from 0.14-1.00 mM/g cellulose, and increased with an increasing COCl:OH ratio of 1-20. For PAA grafted nanofibers, the activity of bound (i.e., adsorbed) lipase decreased with increased COOH on the nanofiber surfaces. In contrast, for PEG grafted nanofibers, the activity of bound (i.e., covalently bound) lipase increased with increased COOH on the nanofiber surfaces. Initial studies of lipase activity for lipase bound to the cellulose nanofiber membranes under various couple reaction conditions is shown in FIG. 14. The efficiency of lipase adsorbed at 0.76 mM COOH/g PAA grafted cellulose is an order of magnitude higher than that of lipase covalently attached to 1.00 mM/g PEG grafted cellulose (391 U/ml vs/41.9 U/ml) (see, FIG. 28). Immobilization of lipase onto the cellulose nanofiber membranes also increased stability of the enzyme when exposed to a variety of organic solvents (see, FIGS. 16 and 29). Lipase covalently bound to PEG-grafted cellulose nanofiber membranes exhibited greater thermal stability than free enzyme (see, FIG. 30). Bound lipases exhibited generally similar pH stability when compared to free lipases (see, FIGS. 16 and 31). Finally, the activity of the bound lipases was measured consecutive enzyme assays using the same membrane. The results demonstrate that membranes with either adsorbed or covalently bound enzymes can be used at least 4 times (see FIGS. 16 and 32).

### Example 3

Generation of Nanofibers Comprising Incorporated Biological Materials

Materials: Polyethylene oxide (PEO) (Ave. M_{w}600,000, Aldrich), polyvinyl alcohol (PVA) (Ave. M_{w}124,000 186,000, Aldrich), casein (Acros), triethanolamine (98%, Aldrich) were used as received. The crosslinking agent 4,4′ methylenebis(phenyl isocyanate) (MDI) from Aldrich was distilled before using. Enzymes of lipase (type VII, from Candida rugosa) and α-Chymotrypsin (type II, from Bovine pancrease) were purchased from Sigma and used as received. The lipase assay regents including olive oil (substrate), gum Arabic (emulsion regent), sodium deoxycholate (emulsion regent), triethanolamine hydrochloride, cryst (buffer), sodium diethyldithiocarbanate (color indicator) and stearic acid (standard) are all from Acros and used without further treatment. Other solvents and buffer regents were obtained from commercial sources and used as received.

Processing: Casein was dissolved in 5% (wt) aqueous triethanolamine at room temperature. PVA and PEO were dissolved in water by heating up to 50° C. and agitating overnight. The lipase was dissolved in 10 mM bis tris propane buffer (pH=7.8) at room temperature. The electrospinning solution was prepared by mixing above solutions at various compositions, and then placed in a glass tube bearing a plastic pipette tip (Fisher, 0.5-10μ1). A stainless steel electrode from a power supply (Gamma High Voltage Research Inc.) was immersed in the solution. A grounded counter-electrode was connected to the collector (aluminum foil). Typically, electrospinning was performed at 25 KV with a 30 cm distance between the plastic tip and the collector. The glass tube was tilted to a slight angle of 2-10 degree from horizontal position to allow a bead of polymer solution at the tip of pipette which acted as the base of jet. To get thick enough membrane that is detachable and integrate, the electrospinning process usually continued for 6 hours or more. For comparison purpose, cast membrane from the same solution as in electrospinning was prepared.

The ES membranes were crosslinked by immersing in 1% MDI in THF for above 10 hours, then washed by THF, acetone and water, and dried by vacuum at room temperature. To study the biodegradability and phase separation of the membranes, the crosslinked ES membrane was digested by α-chymotrypsin (25 mg/ml) in HEPES buffer (pH=7.8) at 25° C. for certain time.

Enzyme Activity Assay: The catalytic activity of the ES membrane containing lipase was assayed based upon a standard photometric method (Schmidt et al, In: Bergmeyer H. U., editor, Methods of Enzymatic Analysis, 2nd English ed., vol. 2., Verlag Chemie: Weinheim, Section C, pp. 819 823 (1974)). Basically, a stabilized olive oil emulsion is used as the substrate. The substrate, lipase immobilized sample and buffer (pH=8.5) were added together and incubated in 30° C. bath shaking at 60 rpm. At designed assaying time (5 hours for solid supported lipase; 10 minutes for free lipase), the incubation solution was heated to 80° C. for 10 minutes to denature the lipase. Copper(II) sulfate aqueous solution and chloroform were added to the incubation tube. The liberated fatty acids by hydrolysis of olive oil were extracted to chloroform layer in the form of their copper salts. The same procedure using denatured lipase was adopted to prepare a blank solution. The amount of copper(II) ion in chloroform, i.e., the amount of COO⁺, was determined spectrophotometrically at 436 nm (HITACHI U 2000 Spectrophotometer) with sodium diethyldithiocarbamate as color indicator. The absorbance was converted to concentration using a calibration from stearic acid standard solution. The amount of free fatty acid liberated per hour under assay condition was a measure of the lipase activity. To trace the time course of enzyme catalyzed hydrolysis, a series of incubation solutions in vials were used. The assay samples were withdrawn from independent vials at different times in order to avoid affecting the enzyme/oil ratio. To measure the activity of recycled lipases, the enzyme membranes after the first assay were washed thoroughly by water, dried in vacuum at room temperature, and stored at 4° C. until the second assay 5 days later.

Other Characterizations: Scanning electronic microscope (SEM) images were collected at 10 kv with International Scientific Instrument model DS 130. Differential scanning calorimetry (DSC) and thermogravimetric analysis (TGA) were performed at a 10° C./min heating rate under N₂ on SHIMADZU DSC-50 and TGA-50, respectively.

Casein ES Membranes. Casein was found to form viscous solutions in 5% (wt) aqueous triethanolamine at 10-30% (wt) concentration. However, these casein solutions could not be electrospun, despite attempts with different solution viscosities, voltage, distance, and other parameters. The casein solutions could not form an equilibrium shape of liquid drop suspended at the tip of the pipette which is necessary to initiate stable jets. Significant die swelling (“Barus effect” in traditional fiber spinning) was observed at the tip of capillary due to the high elasticity of the polymer, which is direct related to the protein structure. However, by adding another polymer of PEO or PVA, casein can be electrospun out successfully. Without intending to be bound by any theory, it is thought that in the case of casein, the addition of the secondary polymer may dissociate the interconnected polypeptide chain of the protein and therefore reduce its elasticity. Electrospinning of both solution mixtures (casein/PVA and casein/PEO) has been carried out at different polymer and protein concentrations and compositions (Table 1). Fine fiber membrane structures can be formed at their optimal conditions. To obtain integrated membrane that is detachable from aluminum foil, the electrospinning process usually lasts for at least 6 hours.

TABLE 1
Electrospinning of PEO/casein and PVA/casein solutions
(a)

| PEO:casein (w:w) | Spinning Observation | Product |
|---|---|---|
| 0:100 | dripping | liquid blot |
| 5:95 | continuous | non fiber membrane, non detachable |
| 20:80 | continuous | non fiber membrane, detachable |
| 40:60 | continuous | — |
| 50:50 | continuous | irregular fiber (w/bead) membrane, |
| | | detachable |
| 60:40 | continuous | — |
| 80:20 | continuous | fine fiber membrane, detachable |
| 100:0 | continuous | fine fiber membrane, detachable |

*5% PEO in water mixed with 5% casein in 5% aq. triethanolamine;
the total polymer concentrations are 5%.

(b)

| PEO:casein (w:w) | Spinning Observation | Product |
|---|---|---|
| 0:100 | dripping | liquid blot |
| 20:80 | continuous | fine fiber membrane, detachable |
| 40:60 | heave jet, blocked after 5 hr | — |
| 50:50 | jet radiation, not easy to collect | — |
| 80:20 | too viscous, no jet come out | — |

*10% PEO in water mixed with 10% casein in 5% aq. triethanolamine;
the total polymer concentrations are 10%.

(c)

| PEO:casein (w:w) | Spinning Observation | Product |
|---|---|---|
| 30:70 | dripping, blocked after 10 min | irregular fiber membrane, non detachable |
| 50:50 | dripping, partially blocked after 30 min | fine fiber membrane, detachable |
| 70:30 | partially blocked after 30 min | fine fiber membrane, detachable |
| 100:0 | partially blocked after 30 min | fine fiber membrane, detachable |

*10% PVA in water mixed with 10% casein in 5% aq. triethanolamine;
the total polymer concentrations are 10%.

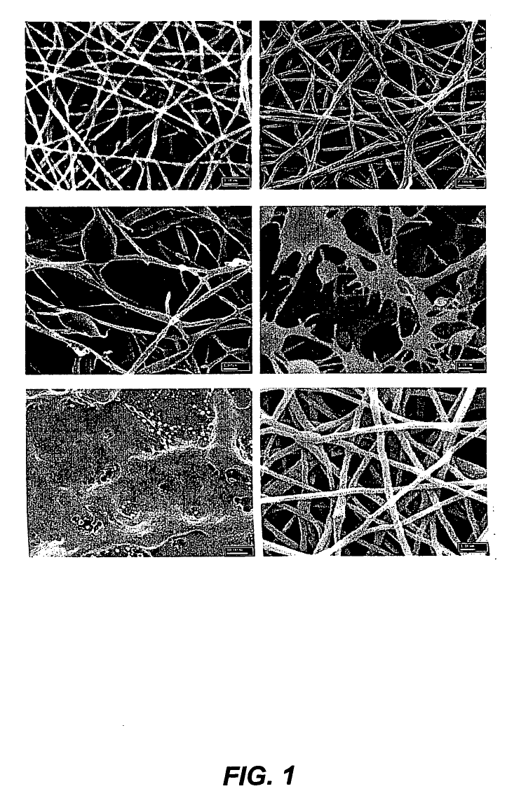

Electrospinning of polymer blend was first carried out with PEO/casein mixture at a 5% total polymer concentration (Table 1-a). PEO is one of the most easily electrospun polymers and has been used as the model polymer to study the processing parameters. The 5% PEO solution itself can be easily electrospun to form fine fibers with diameter generally below 500 nm (FIG. 1-a). By replacing some amount of PEO with casein, the 4:1 (w:w) PEO/casein mixture can still be electrospun into fine fiber membrane (FIG. 1-b). With more casein, the 1:1 PEO/casein mixture is electrospinnable, but the fibers become irregular with large beads (FIG. 1-c). Further increasing casein in the mixture, non fibrous membranes were observed (FIG. 1-d, 1-e). This is in consistence with reported observations that electrospun membranes of PEO range from filamentous membrane to beaded coating with filamentous/beaded intermediate stages when the solution viscosity decreases (see, Deitzel et al., supra; and Fong et al., supra). In the present case, the solution viscosity of 5% PEO was measured at 2791 centipoise, which is much more viscous than the 1.6 centipoise of the 5% casein solution. The viscosity of PEO/casein mixture is mainly from PEO. When PEO is replaced by the same amount of casein, the solution viscosity decreases and the morphologies of ES membranes change accordingly.

When total polymer concentration was increased to 10% (Table 1-b), the polymer solution become more viscous, thus more casein can be included for electrospinning. FIG. 1f shows the SEM image of the ES membrane in which casein reaches 80%. Compared with FIG. 1d at the same PEO/casein ratio, much finer and more uniform fibrous structure was observed at the 10% polymer concentration. However, by pushing PEO/casein ratio higher to the 10% concentration, the viscosity increases dramatically and become difficult to process. (See, FIG. 1).

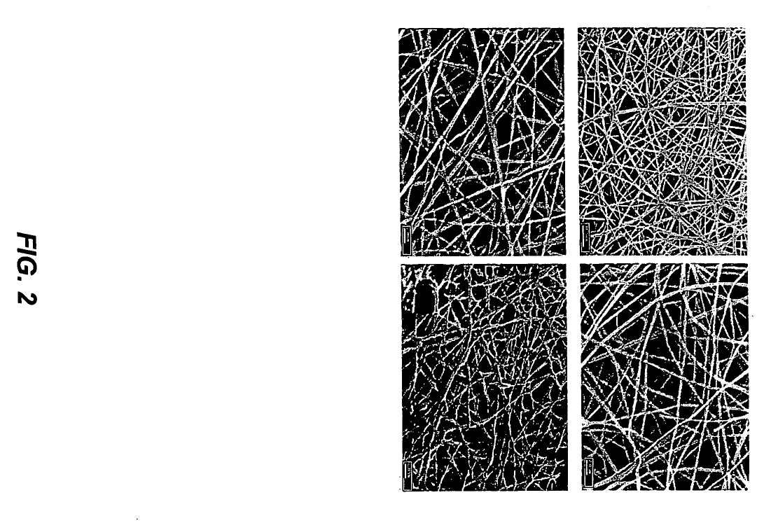

Electrospinning of PVA/casein solution (Table 1-c) is relatively difficult compared with that of PEO/casein solution due to the easily blocked pipette tip. It is necessary to clean the tip from time to time to resume the spinning. FIG. 2 shows the SEM micrographs of PVA/casein membranes. Fine fibrous membranes can be produced at up to 50% casein. As casein reaches above 50%, solution viscosity is too low and irregular fiber structure is formed. (See, FIG. 2).

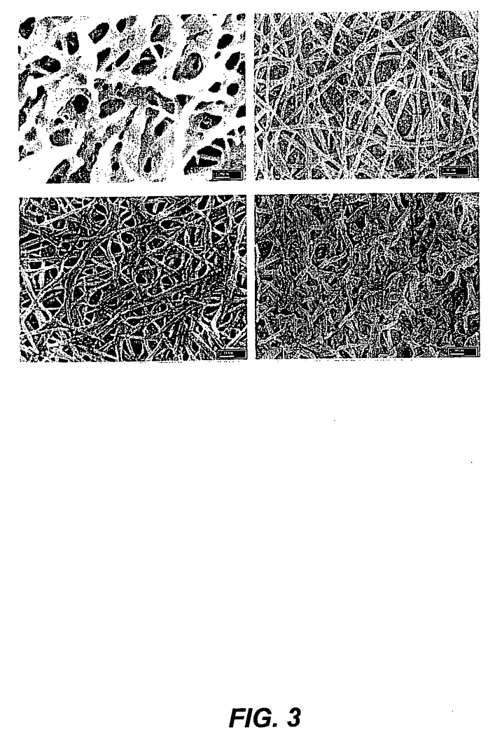

The PEO/casein and PVA/casein as spun fiber membranes are instantly soluble in water. Therefore, they have to be stabilized in order to function in aqueous environment. Diisocynate can crosslink the amine groups on casein and hydroxyl groups on PVA and PEO, making them insoluble in water. The SEM images (FIG. 3) show that the fibrous structure is maintained after crosslinking, although in some environments, the fibers become more densely packed and less stretched. The effects may come from the swelling of fibers by organic solvents and water during and after crosslinking. Although PEO has limited number of —OH as end groups, it can be effectively stabilized and doesn't lose after the crosslinked membranes are thoroughly washed by water, which is convinced by FTIR and TGA analysis. Besides chemical bonding, it is assumed that the physical interaction, especially hydrogen bonding, between PEO and casein molecules also contribute to the strong immobilization of PEO to the substrate. (See, FIG. 3).

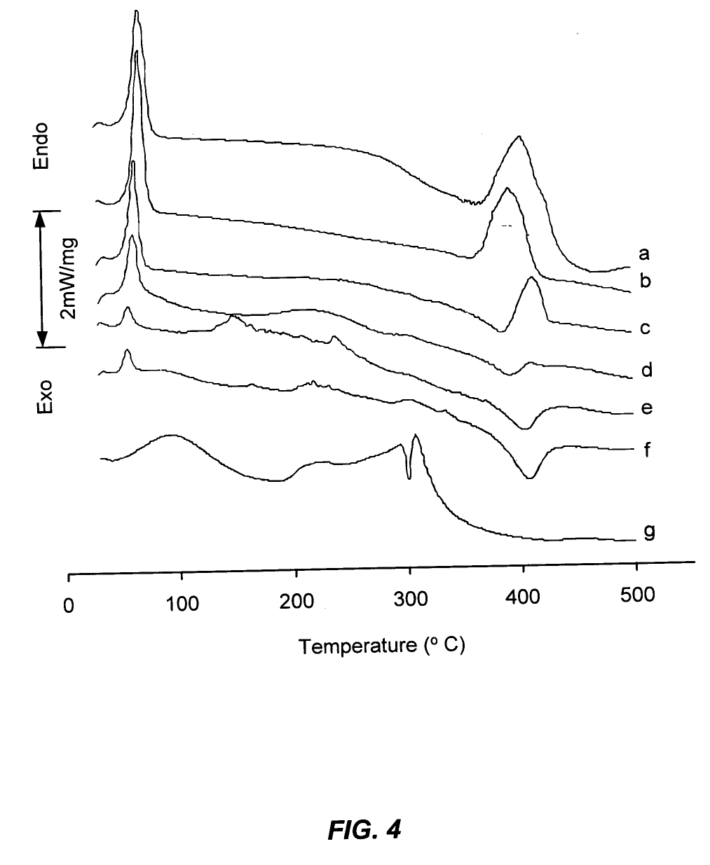

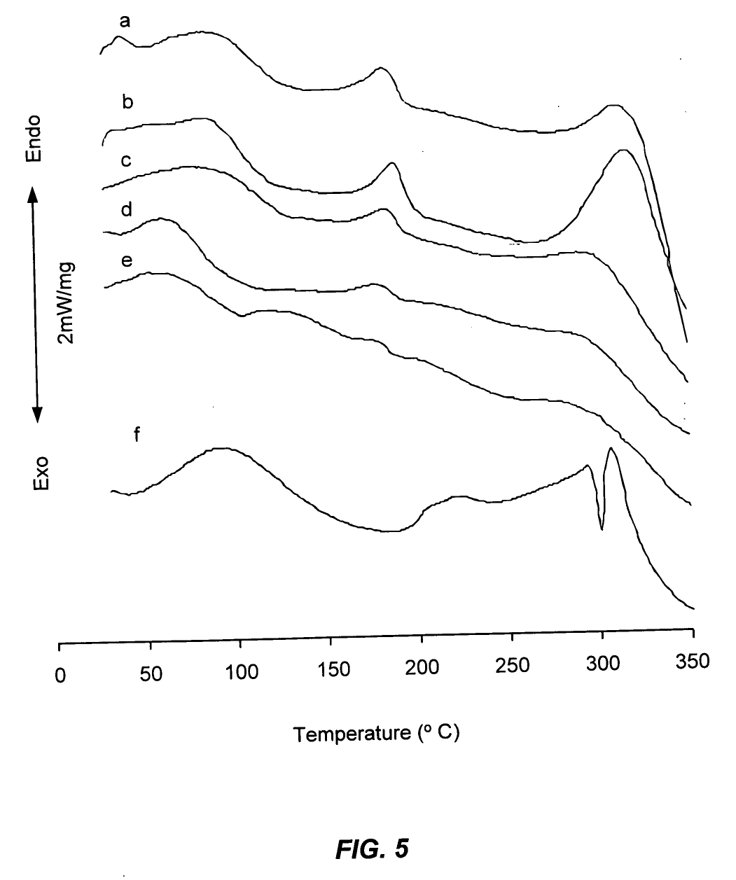

Structure Analysis. DSC analysis of the as spun fiber membranes of PEO/casein series and PVA/casein series are illustrated in FIGS. 4 and 5, respectively. The melting point (Tₘ) and heat of fusion (ΔH_{f}) for PEO and PVA are set forth in Table 2.

Compared with the cast membranes from same solutions, the ES membranes exhibit higher ΔH_{f} for PEO and PVA by 5% and 19%, respectively. The Tₘ for the ES PEO doesn't change significantly, but the ES PVA is increased by 2° C. from cast PVA. Deitzel et al., supra, have studied the crystalline properties of electrospun PEO fibers by DSC and WAXD. The WARD diffraction peaks positions for ES fibers are found identical to those of neat powder, indicating no difference between their crystalline structures. They reported that the crystallinity of ES fibers is much lower than that of neat powder by comparing their diffraction peak intensity, melting points, and heats of fusion. There is no comparison of crystallinity between ES fiber membranes and cast membranes. In the present case, it is evident that the crystallinity of ES membranes is increased from that of cast membrane, which may be induced by the electrical stretching force during spinning. (See, FIGS. 4 and 5).

The DSC data in Table 2 also shows that Tₘ and ΔH_{f} for PEO and PVA in their blends decrease with increasing amount of casein. This phenomenon suggests both PEO and PVA interact to certain extent with casein, in other words, are miscible in fibers. The compatibility may come from the strong hydrogen bonding between these polymers.

TABLE 2
Melting point and heat of fusion of PEO
and PVA in ES membranes
(a)

| | Melting Point | Heat of | Heat of |
|---|---|---|---|
| PEO (wt %) | of PEO (° C.) | Fusion (J/g) | Fusion (J/g PEO) |
| 100 | 70.7 | 161.1 | 161.1 |
| (cast) | | | |
| 100 | 70.2 | 168.9 | 168.9 |
| (ES) | | | |
| 80.0 | 64.9 | 102.5 | 128.1 |
| 50.0 | 62.6 | 74.5 | 149.0 |
| 20.0 | 57.0 | 19.1 | 95.5 |
| 20.0 | 56.2 | 18.7 | 93.5 |

(b)

| | Melting Point | Heat of | Heat of |
|---|---|---|---|
| PVA (wt %) | of PVA (° C.) | Fusion (J/g) | Fusion (J/g PVA) |
| 100 | 186.8 | 37.5 | 37.5 |
| (cast) | | | |
| 100 | 188.7 | 44.6 | 44.6 |
| (ES) | | | |
| 70.0 | 185.8 | 17.0 | 24.3 |
| 50.0 | 180.6 | 11.3 | 22.6 |
| 30.0 | 178.5 | 4.5 | 15.0 |

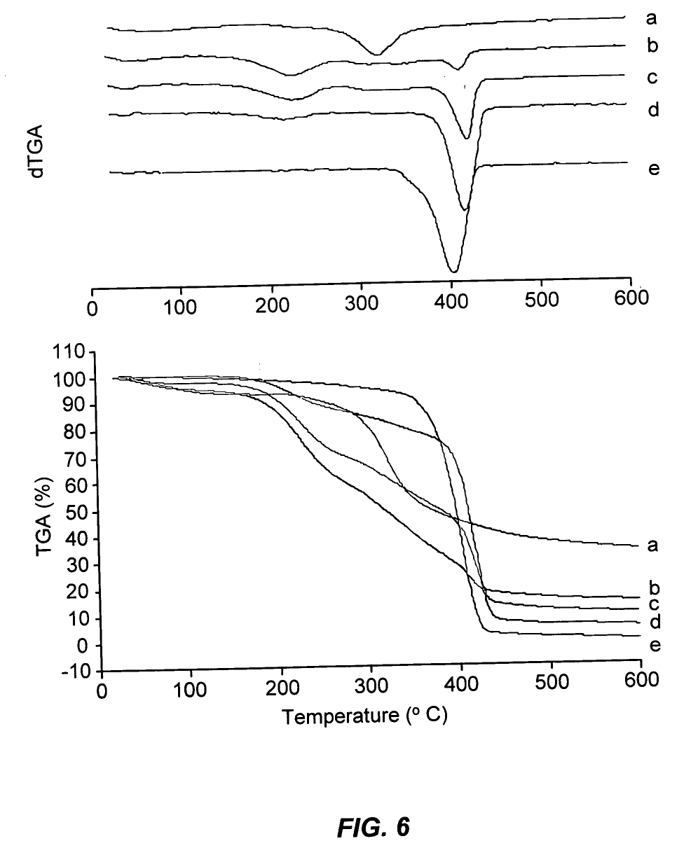

Other evidence also supports the high compatibility and low phase separation in fibers. Even when the as spun membrane of PEO/casein is crosslinked in THF by MDI for only 3 minutes, it becomes insoluble and maintains the membrane shape in water. In contrast, the as spun membranes are highly soluble in water, no matter how long they are stored and dried. The short time treatment is not expected to react all PEO molecules, but may denature the protein (denature) and make it insoluble. It is obvious there is no macroscopic phase separation between PEO and casein. In other words, there is no major domain of PEO on fibers. Otherwise, the membrane shall fall apart in water after PEO is dissolved. An attempt has been made to try to digest casein by a chymotrypsin to study the phase structures on fibers. The two phases are observable after the membrane is digested for 25 hours (FIG. 3-d). However, the fibers are not completely broken. The membrane doesn't fall apart in an aqueous environment, even after 4 days of digestion. Such results are further evidence of the good compatibility between PEO and casein. (See, FIG. 6).

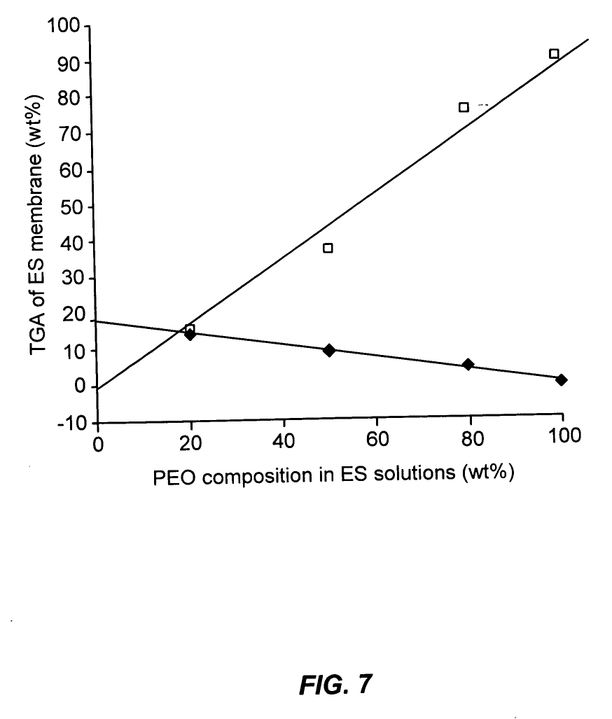

FIG. 6 shows the thermal degradation of PEO/casein membrane. For easy observation of the thermal transition, their derivative curves are also illustrated. The two stage decomposition is obvious. The lower temperature decomposition belongs to casein, while the higher temperature one belongs to PEO. At 600° C., PEO leaves no residue, thus the residue for blend membranes come exclusively from the incomplete decomposition of casein. Because the two decomposition stages do not overlap as illustrated in their derivative curves, the total weight loss of PEO and casein can be obtained separately and used to estimate the composition of PEO and casein in ES membrane. The decomposed PEO in ES membrane is proportional to its theoretical weight composition which is based on the polymer mixing ratio (FIG. 7). The extrapolated trend line pass the original point. The weight of residue at 600° C. is inversely proportional to the mixing ratio of PEO, or proportional to the mixing ratio of casein. Such findings confirm that PEO and casein are electrospun out according to their mixing ratio. There is no major phase separation during electrospinning process (see, FIG. 7).

The thermal decompositions of both PEO and casein are affected by the existence of the other polymer. The decomposition of casein in blend membrane occurs at lower temperature than the decomposition of neat casein powder. This can be explained by the intermolecular forces reduction and regularity destruction of casein after mixing with PEO. On the other hand, the decomposition of PEO is pushed to higher temperatures compared with pure PEO ES membrane. The increased PEO decomposition temperature may result from the strong hydrogen bonding between casein and PEO. It is also possible that the decomposition is suppressed by the nitrogen compound released from casein decomposition.

TABLE 3
Electrospinning of lipase containing solutions
(a)

| PEO:casein:lipase (w:w:w) | Spinning Observation | Product |
|---|---|---|
| 40:40:20 | continuous w/ jet radiation | fine fiber membrane, partially detachable |
| 30:40:30 | continuous w/ jet radiation | fine fiber membrane, partially detachable |

*10% lipase in buffer mixed with 10% PEO in water and 10% casein in
5% aq. triethanolamine; the total polymer concentrations are 10%.

(b)

| PVAaipase (w:w) | Spinning Observation | Product |
|---|---|---|
| 50:50 | jet observable, no collection | — |
| 80:20 | jet observable, collect slowly | fine fiber membrane, partially detachable |

*10% lipase in buffer mixed with 10% PVA in water;
the total polymer concentrations are 10%.

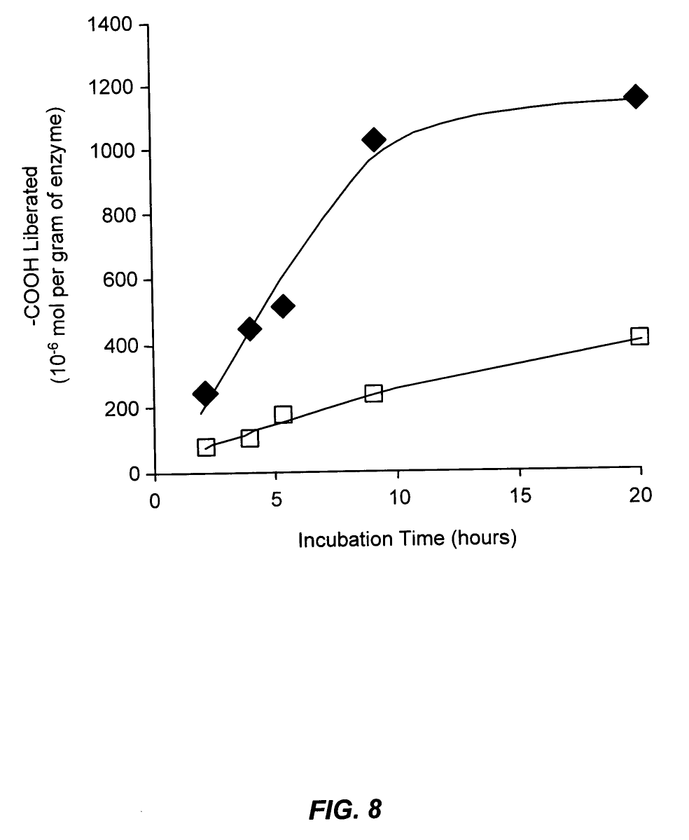

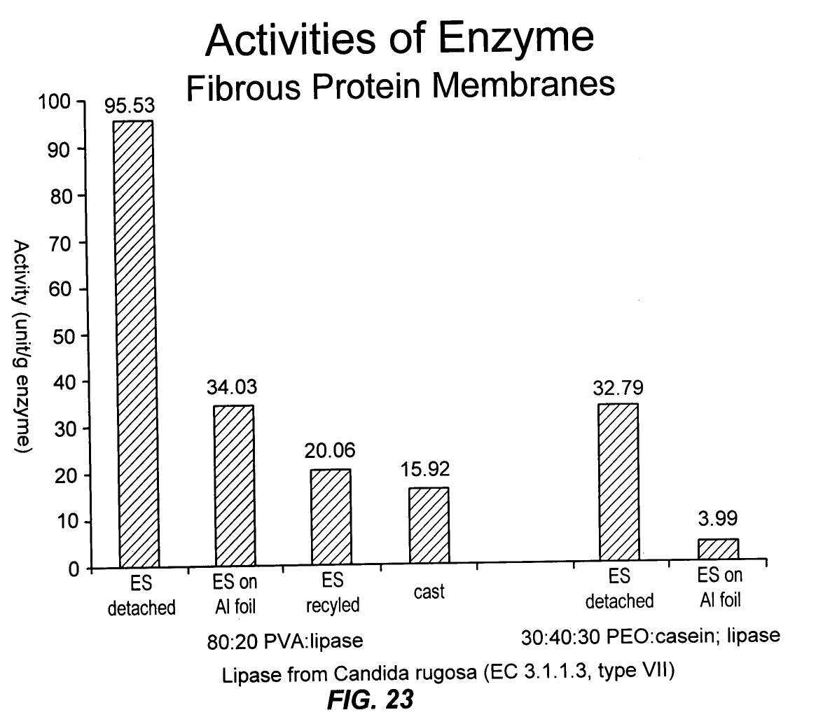

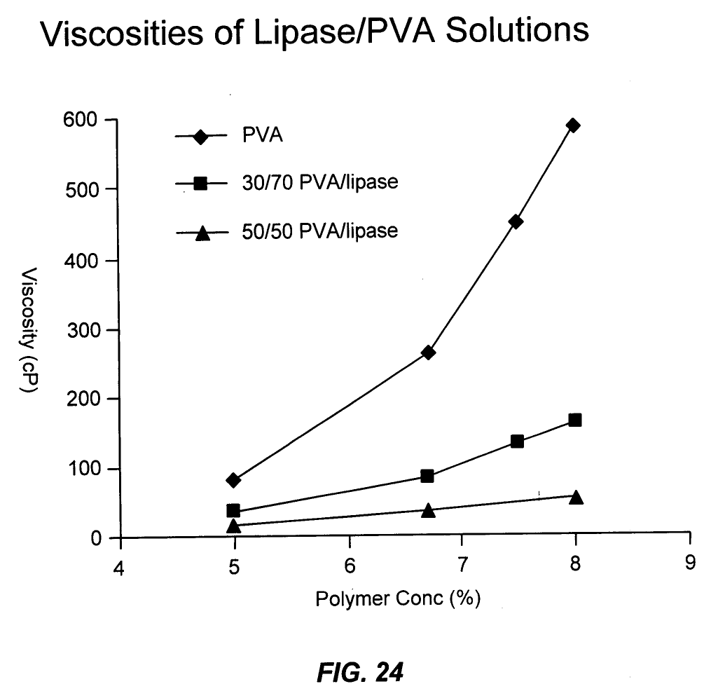

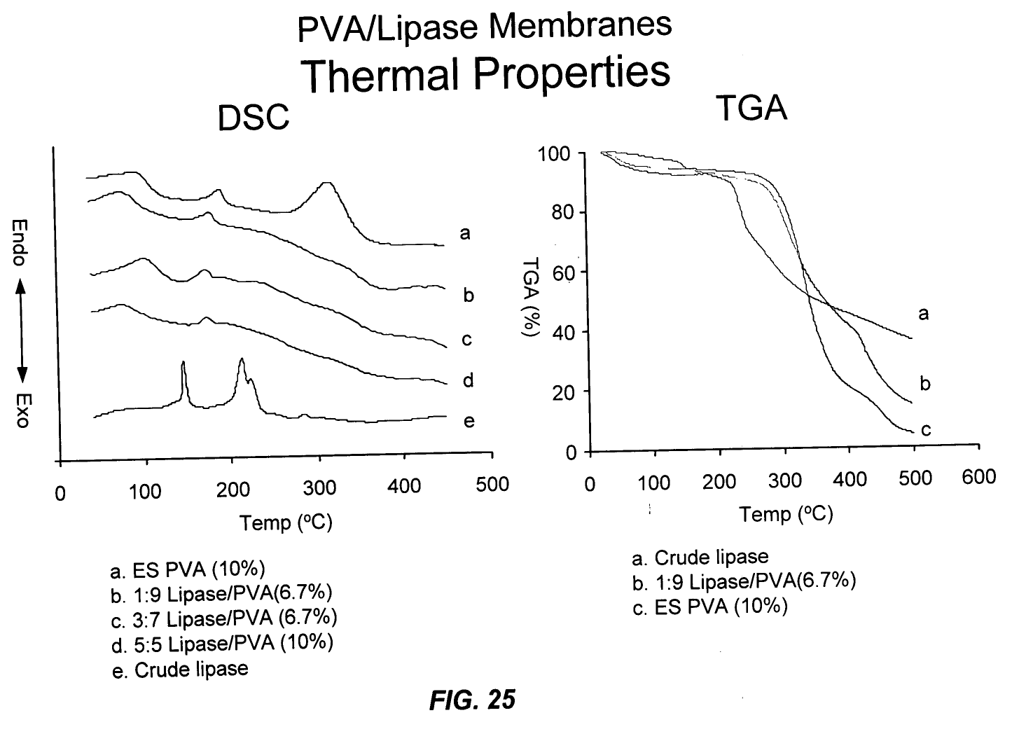

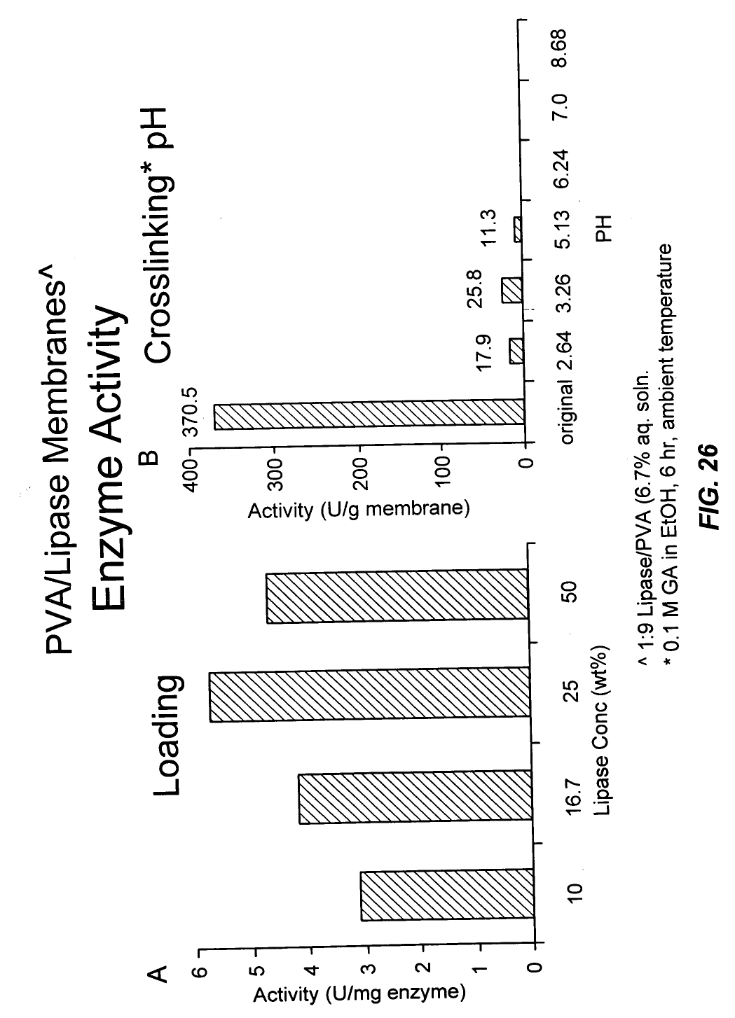

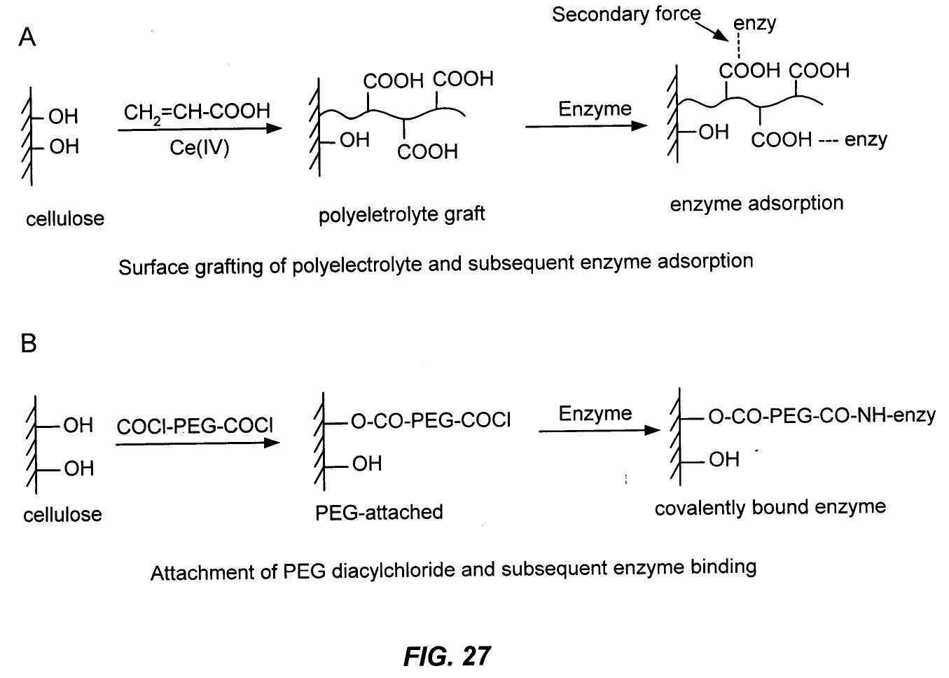

Enzyme carrying ES Membranes. Based on the experiences of processing casein, enzyme carrying ES membranes are prepared by replacing certain amount of casein solution with lipase solution (Table 3). Although both are proteins, their structures and solubility are not expected to be identical, therefore a little variation in polymer composition is made in order to achieve best electrospinning conditions. The lipase containing solutions can be electrospun into fine fibrous membranes which show similar morphology as casein ES membranes. FIG. 8 indicate that during the enzyme catalyzed hydrolysis of olive oil, the liberated —COOH increases linearly with time between 2 and 10 hours, and tends to flat after 10 hours. (See, FIG. 8). The results of addition studies of the activity of lipase bound of PVA and PEO:casein nanofiber membranes are shown in FIG. 23-26.

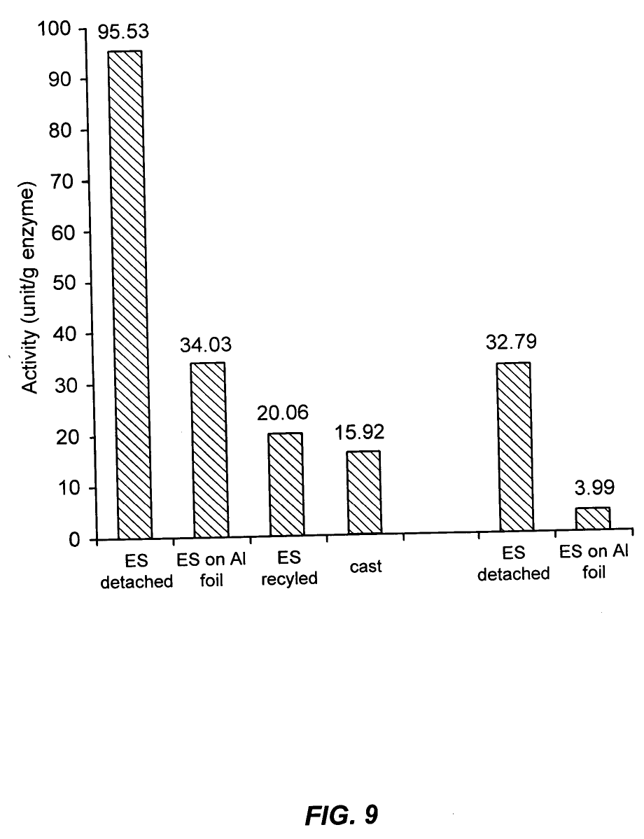

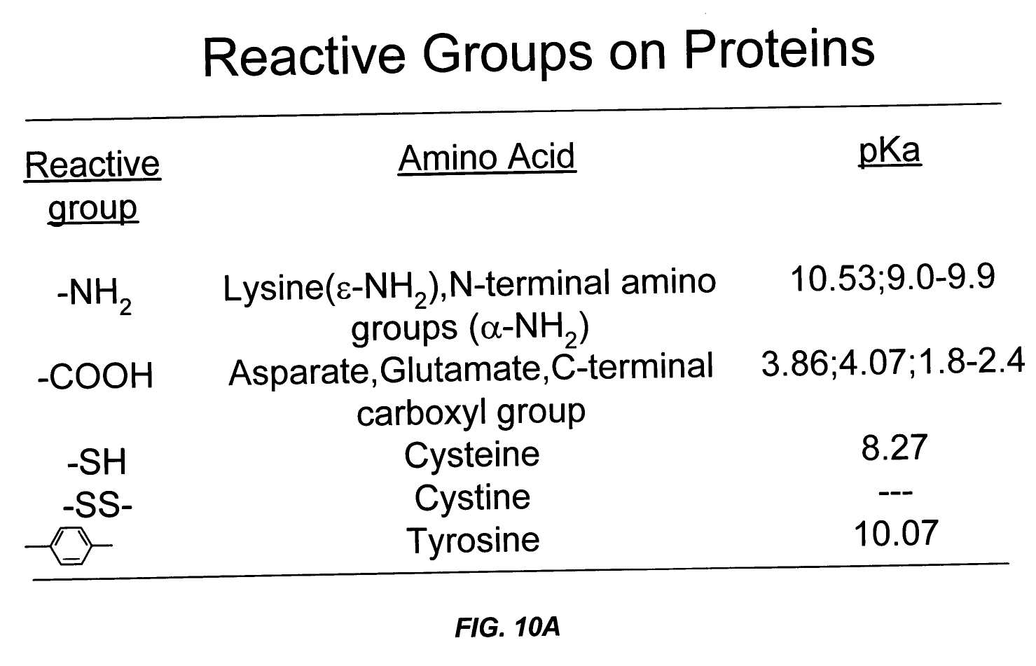

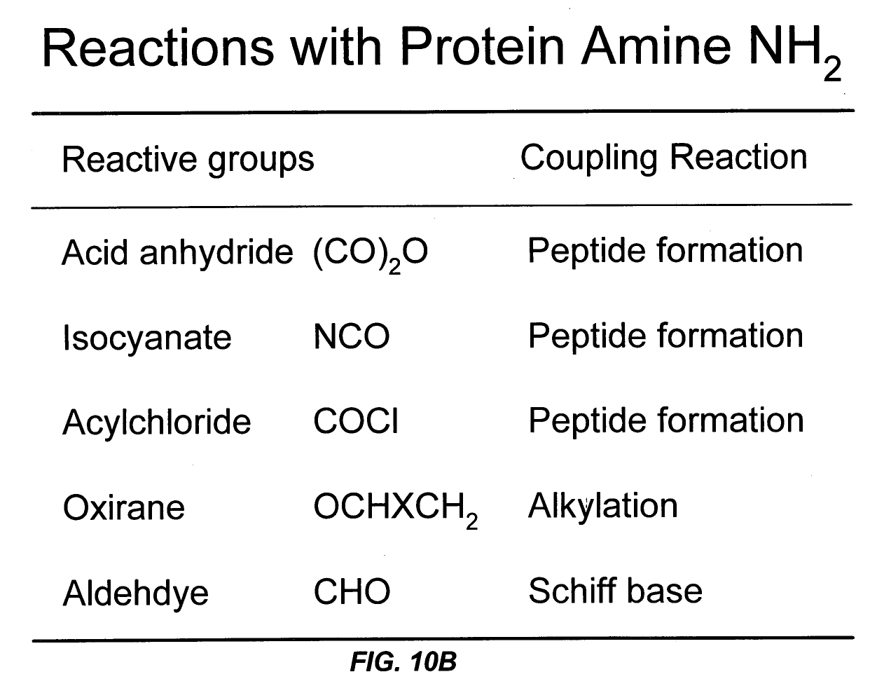

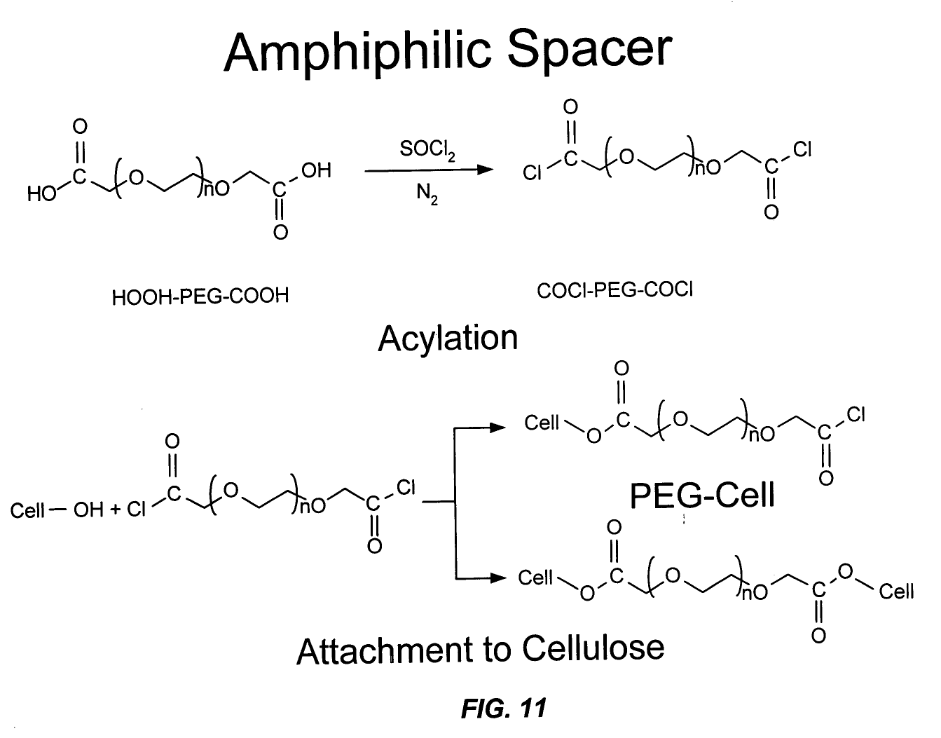

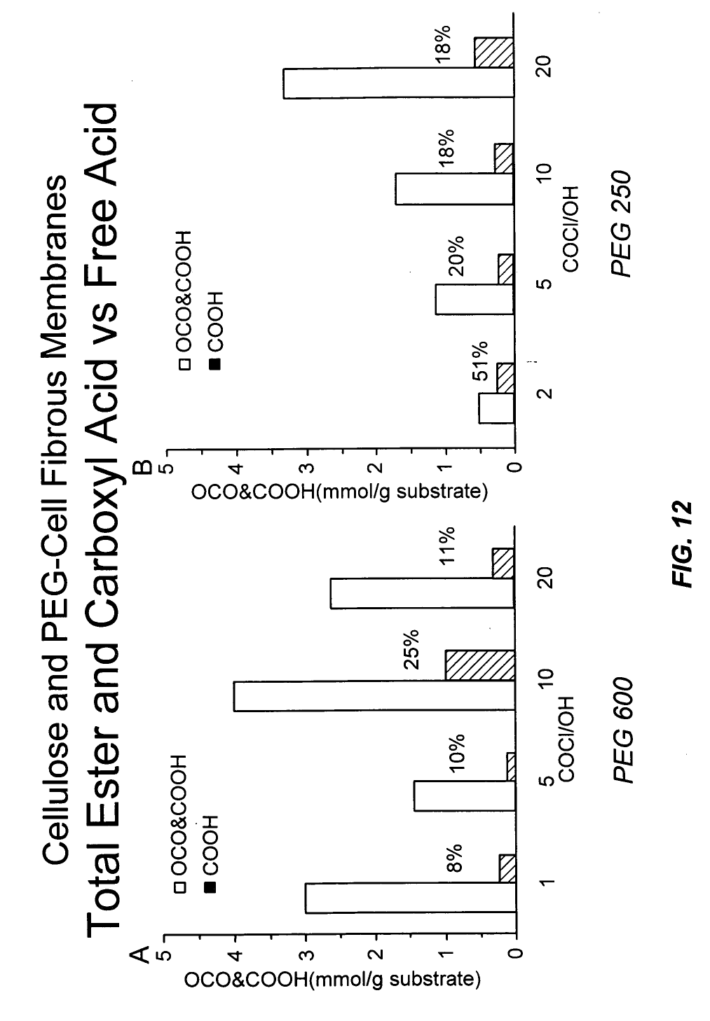

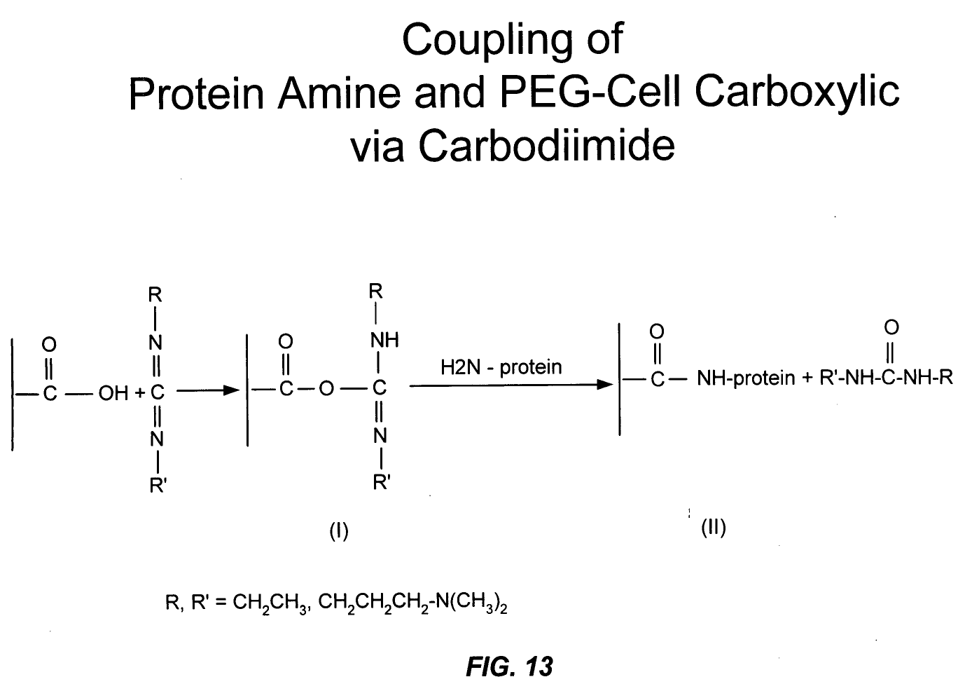

The activities of lipases on different substrates are compared in FIG. 9. The catalytic activity of lipase in ES membrane is about 100 fold lower than that of free lipase. This substantial decrease mainly comes from the inherent difference of the two reaction systems, one catalyst in solid state, the other in soluble state. On the other hand, not all lipases incorporated in polymer blends are exposed to their catalyzing substrate (olive oil) to be involved in the catalytic activity. It is also possible that the lipases are damaged to some extent in electrospinning and stabilization processes. Comparing the two series of lipase carrying membranes, the PVA/lipase membrane exhibit higher catalytic activity than PEO/casein/lipase membrane. Possible reasons include morphology difference, crosslinking effects, polymer interaction, etc., and need further analysis. (See, FIG. 9).

The lipase in PVA/lipase ES membrane is 6 times more active than that in the cast membrane from the same solution. This is consistent with our expectation that electrospun fiber membrane should serve as better enzyme carrying substrate because of their higher surface area and porous structure. If the assay is conducted using ES membrane attached on the collector (aluminum foil), the activity is lower than that of the detached membrane, but still higher than that of cast membrane. The attachment to aluminum foil decreases the total accessible surface area of ES membrane, and makes it less porous. However, this is still in advantage to the cast membrane which has much lower surface area and is totally impenetrable by medium. By attaching to a substrate, it becomes easier to handle and store the ES membranes, and may enhance the enzyme's environmental stability. The method could be further developed by electrospinning the enzyme containing solution onto different collectors, e.g., polymers, metals, or other materials. Numerous enzyme containing composites can be prepared. This could substantially expand the enzyme immobilization approaches.

One advantage of solid supported enzymes is their convenience in repeating usage. After the first cycle of assay, the lipase carrying membrane could be recycled, cleaned, and dried for further usage. The lipase membrane in second round of assay can keep 21% of its original activity. It should be noted there is 5 days of storage time between the two cycles of assays. The activity loss during each step including reaction, recycling, cleaning and storage need further quantitative investigation.

### Example 4

Determination of the Biological Activity of a Biological Material Attached to or Incorporated into a Nanofiber

This example describes methods measure the activities of a biological material (e.g.,lipase) attached to or incorporated into a nanofiber. Polymer based nanofibers comprising lipase are prepared using any one of the methods described above. To measure the lipase activity, triglyceride emulsion is prepared by emulsifying 5 g olive oil in 95 ml NaCl (0.89%) solution using gum arabic as emulsion reagent for 10 min. The incubation mixture is prepared by mixing olive suspension, 10 mM deoxycholate and 1 M triethanolamine buffer (pH 8.5) at the volume ratio of 50:5:45, final concentration being 30 mM, 0.5 mM and 0.5 M, respectively. Lipase activity is measured at 30° C. and pH 8.5. Immobilized lipase is added to 1.0 ml incubation mixture, incubated in a 30° C. bath equipped with shaker and denatured by heating for 10 min at 90° C. 5.0 ml chloroform and 2.5 ml copper reagent are added and mixed in a shaker. The mixture is centrifuged for 5 min to separate the phases and the aqueous phase is removed. To assay the enzyme, 2.0 ml chloroform layer is mixed with 0.25 ml of 11 mM diethyldithiocarbamate. Photometric determination is performed at 440 or Hg 436 nm at ambient temperature against the corresponding sample blank that can be prepared in the same procedure except that enzyme is not activated before the assay.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. A nanofiber comprising a first polymer and a biological material, wherein said nanofiber has a plurality of nanopores.

2. The nanofiber of claim 1, wherein said first polymer is a synthetic polymer.

3. The nanofiber of claim 1, wherein said first polymer is a naturally occurring polymer.

4. The nanofiber of claim 2, wherein said synthetic polymer is a member selected from the group consisting of: poly(ethylene oxide), poly(vinyl alcohol), poly(ethylene naphthalate), polyaniline, polyacrylic acid, polyacrylon nitrile, polystyrene, polymethylmethacrylate, poly(N-isopropylacrylamide), polyvinyl acetate, and derivatives thereof.

5. The nanofiber of claim 3, wherein said naturally occurring polymer is a member selected from the group consisting of: polysaccharides, polypeptides, cellulose, poly-L-lactide, cellulose, casein, and derivatives thereof.

6. The nanofiber of claim 1, wherein said biological material and said first polymer are present in a ratio of about 1:20 to about 20:1.

7. The nanofiber of claim 1, wherein said biological material and said first polymer are present in a ratio of about 1:10 to about 10:1.

8. The nanofiber of claim 1, wherein said biological material and said first polymer are present in a ratio of about 1:5 to about 5:1.

9. The nanofiber of claim 1, wherein said biological material and said first polymer are present in a ratio of 1:4.

10. The nanofiber of claim 1, wherein said biological material is covalently attached to said nanofiber via a linker.

11. The nanofiber of claim 10, wherein said linker is a member selected from the group consisting of: polyethylene glycol (PEG), polyacrylic acid (PAA), polyacrylamide (PAM) as non-ionic, and dimethylaminoethyl methacrylate (DMAEMA) or combinations thereof.

12. The nanofiber of claim 1, wherein said nanofiber is about 50 nm to about 1000 nm in diameter.

13. The nanofiber of claim 1, wherein said nanopores are about 5 nm to about 500 nm in diameter.

14. The nanofiber of claim 1, wherein said nanopores are about 25 nm to about 100 nm in diameter.

15. The nanofiber of claim 1, wherein said nanopores are about 5 nm to about 25 nm in diameter.

16. The nanofiber of claim 1, wherein said nanopores are about 10 nm to about 50 nm in diameter.

17. The nanofiber of claim 1, wherein said nanofiber is insoluble in an aqueous solution.

18. The nanofiber of claim 1, wherein said nanofiber is insoluble in an organic solution.

19. The nanofiber of claim 18, wherein said first polymer is crosslinked.

20. The nanofiber of claim 1, further comprising a second polymer.

21. The nanofiber of claim 20, wherein said first polymer and said second polymer are present in a ratio of about 1:20 to about 20:1.

22. The nanofiber of claim 20, wherein said first polymer and said second polymer are present in a ratio of about 1:10 to about 10:1.

23. The nanofiber of claim 20, wherein said first polymer and said second polymer are present in a ratio of 4:1.

24. The nanofiber of claim 20, wherein said first polymer and said second polymer are present in a ratio of 1:4.

25. The nanofiber of claim 20, wherein said first polymer and said second polymer are present in a ratio of 1:1.

26. The nanofiber of claim 20, wherein said first polymer is a synthetic organic polymer and said second polymer is a naturally occurring polymer.

27. The nanofiber of claim 1, wherein said biological material is a protein.

28. The nanofiber of claim 27, wherein said protein is a member selected from the group consisting of: integral membrane proteins, structural proteins, intracellular proteins, and enzymes.

29. The nanofiber of claim 26, wherein said synthetic organic polymer is a member selected from the group consisting of: poly(ethylene oxide), poly(vinyl alcohol), poly(ethylene naphthalate), polyaniline, polyacrylic acid, polyacrylon nitrile, polysaccharides, cellulose, poly-L-lactide, polystyrene, polymethylmethacrylate, poly(N-isopropylacrylamide), polyvinyl acetate and derivatives thereof, and said naturally occurring polymer is a member selected from the group consisting of: polysaccharides, polypeptides, cellulose, poly-L-lactide, cellulose, casein, and derivatives thereof.

30. The nanofiber of claim 28, wherein said protein is an enzyme.

31. The nanofiber of claim 30, wherein said enzyme is a member selected from the group consisting of: a lipase, a carbohydrolase, a DNAse, and a protease.

32. A membrane comprising a nanofiber comprising a first polymer and a biological material, wherein said nanofiber has a plurality of nanopores.

33. The membrane of claim 32, wherein said membrane is insoluble in an aqueous solution.

34. The membrane of claim 32, wherein said membrane is insoluble in an organic solution.

35. The membrane of claim 32, wherein said biological material is attached to said membrane via a linker.

36. The membrane of claim 35, wherein said linker is PEG.

37. The membrane of claim 35, wherein said linker is PAA.

38. A fabric comprising a nanofiber comprising a first polymer and a biological material, wherein said nanofiber has a plurality of nanopores.

39. The fabric of claim 38, wherein said biological material is attached to said nanofiber via a linker.

40. The fabric of claim 38, wherein said linker is PEG.

41. The fabric of claim 38, wherein said linker is PAA.

42. An insoluble nanofiber comprising a polymer and a biological material, wherein said nanofiber is insoluble in an aqueous solution.

43. An insoluble nanofiber comprising a polymer and a biological material, wherein said nanofiber is insoluble in an organic solution.
